SEQUENCE LISTING (23 sequences)

SEQ ID NO: 1 - protein, 128 aa, synthetic construct
  REGION 1..128: note = Synthetic construct
KRNKPGKATGKGKPVNNKWLNNAGKDLGSPVPDRIANKLRDKEFKSFDDFRKKFWEEVSKDPELSKQFSRNNNDRMKVGKAPKTRTQDVSGKRTSFELHHEKPISQNGGVYDMDNISVVTPKRHIDIH

SEQ ID NO: 2 - protein, 128 aa, synthetic construct
  REGION 1..128: note = Synthetic construct
KRNKPGKATGKGKPVGDKWLDDAGKDSGAPIPDRIADKLRDKEFKNFDDFRKKFWEEVSKDPDLSKQFKGSNKTNIQKGKAPFARKKDQVGGRERFELHHDKPISQDGGVYDMNNIRVTTPKRHIDIH

SEQ ID NO: 3 - protein, 128 aa, synthetic construct
  REGION 1..128: note = Synthetic construct
KRNKPGKATGKGKPVGDKWLDDAGKDSGAPIPDRIADKLRDKEFKNFDDFRRKFWEEVSKDPELSKQFNPGNKKRLSQGLAPRARNKDTVGGRRSFELHHDKPISQDGGVYDMDNLRITTPKRHIDIH

SEQ ID NO: 4 - protein, 128 aa, synthetic construct
  REGION 1..128: note = Synthetic construct
KRNKPGKATGKGKPVGDKWLDDAGKDSGAPIPDRIADKLRDKEFKSFDDFRKAVWEEVSKDPELSKNLNPSNKSSVSKGYSPFTPKNQQVGGRKVYELHHDKPISQGGEVYDMDNIRVTTPKRHIDIH

SEQ ID NO: 5 - protein, 72 aa, synthetic construct
  REGION 1..72: note = Synthetic construct
GGTSDGLEVLFQGPNGEDNQSNHHHHHHHHNQAENSAANNASDDGNQSSNESSSDGEQANTDSAHEGGTAGS

SEQ ID NO: 6 - protein, 201 aa, synthetic construct
  REGION 1..201: note = Synthetic construct
GGTSDGLEVLFQGPNGEDNQSNHHHHHHHHNQAENSAANNASDDGNQSSNESSSDGEQANTDSAHEGGTAGSKSNEPGKATGEGKPVNNKWLNNAGKDLGSPVPDRIANKLRDKEFESFDDFRETFWEEVSKDPELSKQFSRNNNDRMKVGKAPKTRTQDVSGKRTSFELNHQKPIEQNGGVYDMDNISVVTPKRNIDIEG

SEQ ID NO: 7 - protein, 199 aa, synthetic construct
  REGION 1..199: note = Synthetic construct
GGTSDGLEVLFQGPNGEDNQSNHHHHHHHHNQAENSAANNASDDGNQSSNESSSDGEQANTDSAHEGGTAGSKSNEPGKATGEGKPVGDKWLDDAGKDSGAPIPDRIADKLRDKEFENFDDFRETFWEEVSDPDLSKQFKGSNKTNIQKGKAPFARKKDQVGGRERFELNHDKPIEQDGGVYDMNNIRVTTPKRNIDIN

SEQ ID NO: 8 - protein, 200 aa, synthetic construct
  REGION 1..200: note = Synthetic construct
GGTSDGLEVLFQGPNGEDNQSNHHHHHHHHNQAENSAANNASDDGNQSSNESSSDGEQANTDSAHEGGTAGSKSNEPGKATGEGKPVGDKWLDDAGKDSGAPIPDRIADKLRDKEFENFDDFRETFWEEVSKDPELSKQFNPGNKKRLSQGLAPRARNKDTVGGRRSFELNHDKPIEQDGGVYDMDNLRITTPKRNIDIN

SEQ ID NO: 9 - protein, 203 aa, synthetic construct
  REGION 1..203: note = Synthetic construct
GGTSDGLEVLFQGPNGEDNQSNHHHHHHHHNQAENSAANNASDDGNQSSNESSSDGEQANTDSAHEGGTAGSEEERENNPGKATGEGKPVGDKWLDDAGKDSGAPIPDRIADKLRDKEFESFDDFREAVWEEVSKDPELSKNLNPSNKSSVSKGYSPFTPKNQQVGGRKVYELNHDQPMWQGGEVYDMDNIRVTTPRRNIEIG

SEQ ID NO: 10 - protein, 87 aa, synthetic construct
  REGION 1..87: note = Synthetic construct
MELKNSISDYTEAEFVQLLKEIEKENVAATDDVLDVLLEHFVKITEHPDGTDLIYYPSDNRDDSPEGIVKEIKEWRAANGKPGFKQG

SEQ ID NO: 11 - protein, 85 aa, synthetic construct
  REGION 1..85: note = Synthetic construct
MELKASISDYTEAEFLQLVTTICNADTSSEEELVKLVTHFEEMTEHPSGSDLIYYPKEGDDDSPSGIVNTVQQWRAANGKSGFKQ

SEQ ID NO: 12 - protein, 356 aa, synthetic construct
  REGION 1..356: note = Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGDTSGTGAEGRRGEEAGKHHHHHHHHGKGAEEGRRGEAGLEVLFQGPGQASGTTMGKIEEGKQETWNNGDKGYNGRAEDGGCGGAIEELEEEVRRHQMRLMALQLEEQLMGGCGDVGDMEFRNSISDYTEEEFVRLLRGIERENVAATDDRLDWMLEHFVEITEHPDGTDLIYYPSDNRDDSPEGIVEEIREWREANGRPGFKQGGSTDGGDVGEERNRRCEELNEEIEEHRERLRQLEETREECRT

SEQ ID NO: 13 - protein, 354 aa, synthetic construct
  REGION 1..354: note = Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGDTSGTGAEGRRGEEAGKHHHHHHHHGKGAEEGRRGEAGLEVLFQGPGQASGTTMGKIEEGKQETWNNGDKGYNGRAEDGGCGGAIEELEEEVRRHQMRLMALQLEEQLMGGCGDVGDMEFRDSISDYTEEEFLRLVTSICNADTSSEEELVWMVTHFEEITEHPSGSDLIYYPREGDDDSPSGIVNTVRQWREANGRSGFQQGSTDGGDVGEERNRRCEELNEEIEEHRERLRQLEETREECRT

SEQ ID NO: 14 - DNA, 6361 bp, synthetic construct
  misc_feature 1..6361: note = Synthetic construct
  source 1..6361: mol_type = other DNA
acgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaaccttaagaaggagatataccatgagcgataaaattattcacctgactgacgacagttttgacacggatgtactcaaagcggacggggcgatcctcgtcgatttctgggcagagtggtgtggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggcgggtgacactagtggtaccatggtcgacggtactgcaggttccaaaagcaacgaaccgggtaaagcaaccggtgagggtaaaccggttaacaacaaatggctgaacaacgccggtaaagacctgggttccccggttcctgaccgcatcgcgaataagctgcgcgataaggaatttgaatcctttgacgatttccgtgaaaccttttgggaagaagtctctaaagacccggaactgagcaaacagttctcccgcaacaacaacgaccgcatgaaagtgggcaaagctccgaaaacccgtactcaagacgtttctggcaaacgcactagctttgaactgaaccatcagaagccgattgaacaaaacggcggtgtttatgatatggacaacatctccgtggttacgccgaaacgtaacattgacatcgaaggtactgcaggtcacgtcggtgatggttctctgcaagattctgaagttaatcaggaagctaaaccggaagttaaaccggaagttaaaccagaaactcacattaacctgaaagtgagcgatggttcttccgaaatctttttcaagattaaaaagactactccgctgcgtcgtctgatggaagcattcgcgaaacgccaaggtaaagaaatggattctctgcgctttctgtatgacggcattcgcattcaggctgaccaagcaccggaggatctggacatggaagataatgacattattgaagctcatcgtgaacagatcggtggcagcactggtctggaagtcctgttccaaggcccgggtcaagcttcttaatgactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccataccc

SEQ ID NO: 15 - DNA, 6361 bp, synthetic construct; identical to SEQ ID NO: 14
  misc_feature 1..6361: note = Synthetic construct
  source 1..6361: mol_type = other DNA

SEQ ID NO: 16 - DNA, 5976 bp, synthetic construct
  misc_feature 1..5976: note = Synthetic construct
  source 1..5976: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcaaaatcgaagaaggtaaacaggaaacctggaataacggcgataaaggctataacggtcgtgctgaagacggtctggaagtcctgttccaaggcccaggtcaagcttctagcggccgcgaaggtactagtgatggcctggaagtactgtttcaaggtccgaacggtgaagataaccagagcaaccatcaccaccatcatcatcaccacaatcaggctgaaaactctgccgcaaacaatgcgtctgacgacggtaaccagagctctaacgagtcttcttccgatggcgaacaggcaaacaccgactctgcacacgaaggtggcaccgcaggttccaaaagcaacgaaccgggtaaagcaactggtgagggtaaaccagttaacaacaaatggctgaacaacgccggtaaagacctgggttccccggttcctgaccgcatcgcgaataagctgcgcgataaggaatttgaatcctttgacgatttccgtgaaaccttttgggaagaagtctctaaagacccggaactgagcaaacagttctcccgcaacaacaacgaccgcatgaaagtgggcaaagctccgaaaacccgtactcaagacgtttctggcaaacgcactagctttgaactgaaccatcagaagccgattgaacaaaacggcggtgtttatgatatggacaacatctccgtggttacgccgaaacgtaacatcgatatcgaaggttaattaagctgaacaagctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 17 - DNA, 6678 bp, synthetic construct
  misc_feature 1..6678: note = Synthetic construct
  source 1..6678: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgaactatatgaaacgtcgtctgctgctgttcgcaggtatcctgctgctggtagctctggcaggttcttctactacggatccaattacctctgaatctgaaggcatttggaaccactttttcgtatatccgatgtcttggctgattactacggtggcgaacctgctgaacggtagctacggtctgtccattatcattgtcaccatcctgattcgtctggctctgctgcctctgacgctgaaacagcaaaaaagcatgcgtgccatgcaggtgattcgccctgaaatggaagctattcagaaaaagtacaaagagaaaggttctaaagatcctaaggtacagcaagaaatgcagaaagaactgctgggtctgtaccagaaacacggtgtaaacccgatggctggctgtctgcctctgtttatccaactgccgattctgatggcgttctactttgcgattatgcgtacggaagagattcgttatcatacgtttctgtggtttgatctgggtcaacctgactacattctgccttttgttgctggcatcactacgtactttcagttcaaaatgacgatgagccatcagcaacagatgcagaaaacgaatccgtccgattccgataacccaatggcgaacatgatgcagatgcagatgaaagtgatgctgtatgtaatgccggttatgatcatcatcgcgggtctgtccctgccatccgctctgtctctgtactgggtgatcggcaacatctttatgattattcagacgtactttatcgtcgttaaagcaccaccgctggaagtcgaacagaccaaacaaaaatcttccggtggcactagtgatggcctggaagtactgtttcaaggtccgaacggtgaagataaccagagcaaccatcaccaccatcatcatcaccacaatcaggctgaaaactctgccgcaaacaatgcgtctgacgacggtaaccagagctctaacgagtcttcttccgatggcgaacaggcaaacaccgactctgcacacgaaggtggcaccgcaggttccaaaagcaacgaaccgggtaaagcaactggtgagggtaaaccagttaacaacaaatggctgaacaacgccggtaaagacctgggttccccggttcctgaccgcatcgcgaataagctgcgcgataaggaatttgaatcctttgacgatttccgtgaaaccttttgggaagaagtctctaaagacccggaactgagcaaacagttctcccgcaacaacaacgaccgcatgaaagtgggcaaagctccgaaaacccgtactcaagacgtttctggcaaacgcactagctttgaactgaaccatcagaagccgattgaacaaaacggcggtgtttatgatatggacaacatctccgtggttacgccgaaacgtaacatcgatatcgaaggttaattaagctgaacaagctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 18 - DNA, 7752 bp, synthetic construct
  misc_feature 1..7752: note = Synthetic construct
  source 1..7752: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaaccttaagaaggagatataccatgaactatatgaaacgtcgtctgctgctgttcgcaggtatcctgctgctggtagctctggcaggttcttctactaccgatgcagatgctagcggcaaacatcaccatcaccatcaccatcacaaatctcaagcttctggtctgccagaaactggtgaaaacggtgatacggatcctgacggtcatgatggtcatgatgacgatgtgattgatatcgaggatgacctggacgatgtcattgaagaggtagaagactctaaaccagataccactgctcctccttcttctccgaaagttacttacaaagctccagttccaactggcgaagtatattttgctgattcttttgaccgtggcactctgtctggctggattctgtccaaagccaaaaaagacgataccgatgatgaaattgccaaatatgatggcaaatgggaggtagaggaaatgaaagagagcaaactgccaggtgataaaggtctggtcctgatgtctcgcgccaaacatcatgccatctctgctaaactgaacaagccgttcctgtttgacaccaaacctctgattgttcagtatgaggttaatttccaaaatggtattgaatgtggtggtgcctatgtgaaactgctgtctaaaactccagaactgaacctggatcagttccatgacaaaaccccttatacgattatgtttggtccagataaatgtggtgaggactataaactgcacttcatcttccgtcacaaaaacccgaaaacgggtatctatgaagaaaaacatgctaagcgcccagatgcagatctgaaaacctattttactgacaaaaaaactcatctgtacactctgatcctgaatccagataatagctttgaaatcctggttgaccaatctgttgtcaatagcggtaatctgctgaatgacatgactcctcctgtaaatccttctcgtgaaattgaggacccagaagaccgcaaaccggaggattgggatgaacgtccaaaaatcccagatccagaagctgtcaaaccagatgactgggatgaagatgcccctgctaaaattccggatgaagaggccactaaaccggaaggctggctggatgatgaacctgagtacgtacctgatccagacgcagagaaacctgaagactgggacgaagacatggatggtgaatgggaagctcctcagattgccaaccctcgttgtgagtctgctcctggttgtggtgtctggcagcgtcctgtgattgacaacccgaattacaaaggcaaatggaaacctcctatgattgacaatccgagctaccagggtatctggaaaccgcgtaaaatcccaaatccagatttctttgaagatctggaacctttccgtatgactccttttagcgctattggtctggaactgtggtccatgacctctgacatctttttcgacaactttatcatttgtgctgatcgtcgcattgttgatgattgggccaatgatggttggggcctgaaaaaagctgctgatggtgctgctgaaccaggcgttgtgggtcagatgatcgaagcagctgaagaacgcccgtggctgtgggtagtctatattctgactgtagccctgcctgtgttcctggttatcctgttctgctgcagcggcaaaaaacagacctctggtatggaatacaaaaaaactgatgcacctcaaccggatgtgaaggaagaggaggaagagaaagaggaagaaaaagataaaggtgacgaggaggaggaaggcgaggagaaactggaagagaaacagaaaagcgatgctgaagaagatggtggcactgtcagccaagaagaagaggatcgtaaacctaaagcagaagaagatgaaattctgaacagcggccgcgaaggtactagtaatggtgatggcctggaagtactgtttcaaggtccaggtctgcctgagaccggtaatggtgaagataaccagagcaacggtgctcagaatggtatcgatggcaatcaggctgaaaactctgctgcaaacaatgcgtctgacgacggtaaccagagctctaacgagtcttcttccgatggcgaacaggcaaacaccgactctgcacacgaaggtggcaccgcaggttccaaaagcaacgaaccgggtaaagcaactggtgagggtaaaccagttaacaacaaatggctgaacaacgccggtaaagacctgggttccccggttcctgaccgcatcgcgaataagctgcgcgataaggaatttgaatcctttgacgatttccgtgaaaccttttgggaagaagtctctaaagacccggaactgagcaaacagttctcccgcaacaacaacgaccgcatgaaagtgggcaaagctccgaaaacccgtactcaagacgtttctggcaaacgcactagctttgaactgaaccatcagaagccgattgaacaaaacggcggtgtttatgatatggacaacatctccgtggttacgccgaaacgtaacatcgatatcgaaggttaattaagctgaacaagctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 19 - DNA, 6394 bp, synthetic construct
  misc_feature 1..6394: note = Synthetic construct
  source 1..6394: mol_type = other DNA
gcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaaccttaagaaggagatataccatgagcgataaaattattcacctgactgacgacagttttgacacggatgtactcaaagcggacggggcgatcctcgtcgatttctgggcagagtggtgtggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggcgggtgacactagtggtaccggcgctgaaggccgtcgtggtgaagaagcgggcaaacaccatcaccatcaccatcaccacggtaaaggcgcggaagagggtcgtcgcggtgaagctggcctggaagtcctgttccaaggcccgggtcaagcttctggtaccaccatgggcaaaatcgaagaaggtaaacaggaaacctggaataacggcgataaaggctataacggtcgtgctgaagacggtggttgtggtggtgctattgaagaactggaagaagaagtacgtcgtcaccagatgcgtctgatggccctgcaactggaagaacagctgatgggtggctgtggtgacgtcggtgacatggagttccgtaactctatctctgattacaccgaggaagaatttgttcgcctgctgcgcggtattgagcgcgagaatgttgcggctactgacgaccgtctggactggatgctggaacactttgtcgaaattaccgaacacccagacggcactgacctgatttactatccgtctgataaccgtgatgattctccagaaggcatcgttgaagaaattcgtgagtggcgtgaggcgaatggtcgtcctggcttcaagcagggcggttctaccgatggcggcgacgtcggtgaagaacgtaaccgtcgttgcgaagagctgaacgaagagatcgaggaacaccgtgaacgtctgcgtcagctggaggaaacccgcgaagaatgtcgcaccggcactgctaaaagcgaagagggcaaacaggaaacttctaatcatggtgataaaggtacgaacggtcaggcagaagatagcggccgctaatgactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcga

SEQ ID NO: 20 - DNA, 15883 bp, synthetic construct
  misc_feature 1..15883: note = Synthetic construct
  source 1..15883: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcaaaatcgaagaaggtaaacaggaaacctggaataacggcgataaaggctataacggtcgtgctgaagacggtctggaagtcctgttccaaggcccaggcacctcaggtggcaccatgggcctggattccaagctgaaggcaccagtcttcacggtgcgcacccagggtcgtgagtacggtgagttcgtcctggaaccgctggagcgtggcttcggcgtcaccctgggcaacccactgcgtcgcatcctcctgtcttccatccctggtactgcggtcaccagcgtctacattgaggatgtcctgcacgagttctccaccatcccgggcgttaaagaggacgttgtagagattatcctgaacctgaaggaactggttgttcgtttcctgaacccgagcctgcaaaccgtgaccctgctcctgaaagccgaaggcccaaaggaagttaaggcacgcgacttcctgccagttgccgacgttgagatcatgaacccggacctgcacatcgccaccctggaagagggcggtcgtctgaacatggaggtccgcgttgatcgcggcgttggttacgtcccggcggaaaaacacggcatcaaggaccgtatcaacgccatcccggtggacgccgtcttctccccggtgcgtcgcgtggctttccaggtggaagacactcgcctgggtcaacgcaccgacctggacaagctgaccctccgtatctggaccgacggctccgtcaccccgctggaggccttaaaccaggcggttgagatcctgcgtgagcacctgacctacttctccaacccgcaagcggcagccgttgcggcaccggaagaggctaaagagccggaagcgccgccagagcaggaggaagagctggacctgccgctggaagagcttggcctgtccacccgcgttctgcacagcctgaaggaagagggcattgagtccgtccgcgcactgctggcactgaacctgaaagacctgaagaacatcccaggtattggcgaacgtagcctggaagagatcaaggaagccctggagaagaaaggcttcaccctgaaagagtaatgaatttaaatagtaaatatggcctcaaataacgtaaaaacccgcttcggcgggtttttttatggggggagtttagggaaagagcatttgtcagaatcaagcatcaatgactgcagacttaagaaggagatataccatgagcgataaaattaatcaccagactgacgacagttatgacacggatgtagacaaagcggacggcgcgaaccaggtcgactactccgcagagctggaagtcctgttccaaggccctggtcaagcttctggtgcggacggtggtatggaaatcaaacgtttcggtcgcatccgtgaagttatcccgctgccgccgctgaccgaaatccaggtggaatcctaccgccgtgcgctgcaagccgatgtcccgccggagaaacgtgagaacgtcggcatccaggcggccttccgtgaaaccttcccgattgaagaagaggacaagggcaaaggcggtctggttctggacttcctggaataccgcctgggcgaaccaccgtttccgcaggacgagtgccgtgagaaagacctgacctatcaggctccgctgtacgcccgtctgcaactgatccacaaagacacgggcctgatcaaggaagacgaggtcttcctgggtcacatcccgctgatgaccgaggacggttccttcatcatcaacggtgccgaccgtgtcatcgtttcccagatccatcgctccccgggtgtctacttcaccccagatccggcgcgcccaggtcgctacatcgcctctatcattccgctgccgaaacgtggcccgtggattgacctcgaagtggaaccgaacggcgttgtctccatgaaggtcaacaaacgtaagttcccgctggttctgctgctgcgtgtcctgggttacgaccaggaaaccctggcacgcgagctgggcgcttacggcgagctggtacagggcctgatggacgagtctgtcttcgctatgcgcccggaagaggctctgatccgcctgttcaccctgctgcgcccgggtgatccaccgaaacgtgacaaggctgtggcctacgtctacggcctgatcgctgacccgcgccgttacgacctgggcgaagccggtcgttacaaggcggaagagaaactgggtatccgcctgtctggtcgcaccctggctcgctttgaggacggtgagttcaaagacgaggtcttcctgccgaccctgcgttacctgttcgccctgaccgccggtgttccgggccacgaggtggacgacattgaccatctgggcaaccgccgcatccgcaccgtgggtgagctgatgaccgaccagttccgtgtgggtctggctcgcctggcacgtggtgttcgtgagcgtatgctgatgggctctgaggacagcctgaccccggctaagctggtcaacagccgtccgctggaagccgctatccgtgaattcttcagccgcagccagctgtcccagttcaaagacgagactaacccgctgtcctctctgcgtcacaaacgtcgcatctccgctctgggtccaggtggtctgactcgcgaacgtgcgggttttgacgtgcgcgacgttcaccgcacccactacggtcgcatctgcccggttgaaacccctgaaggcgccaacatcggcctgatcacctccctggcggcttacgcccgtgtggacgagctcggcttcatccgcaccccgtatcgccgtgttgtgggcggtgtggtcaccgacgaagtggtctacatgacggccaccgaagaggaccgctacaccatcgcccaggccaacaccccgctggagggtaaccgtatcgcggcagaacgcgtcgttgctcgtcgcaagggtgaacctgttatcgttagccctgaacaggttgagttcatggatgtctccccgaagcaggtcttctccgtgaacaccaacctgattccgttcctggagcacgacgacgccaaccgtgccctgatgggctccaacatgcagacccaagccgtgccgctgatccgtgcacaggctccggtggtgatgacgggcctggaagagcgtgtcgttcgcgactccctggcagccctgtacgccgaagaggatggcgaagttgccaaggttgacggcaaccgcatcgtcgttcgctacgaagacggtcgcctggttgagtatccgctgcgtcgcttctaccgctccaaccagggtacggccctggatcagcgtccgcgtgtggttgtgggtcagcgtgtgcgcaaaggtgacctgctggctgacggcccagcctccgagaacggcttcctggccctgggtcagaacgtcctggttgccatcatgccgtttgacggttacaactttgaggacgccatcgtcatcagcgaagagctgctgaaacgcgacttctacacctccatccacattgagcgctacgagattgaggctcgtgacaccaaactgggcccggaacgtatcacccgtgacatcccgcacctgtccgaagcagccctgcgtgacctggacgaggaaggcgttgtccgcatcggcgctgaggtcaaaccaggcgacatcctggttggtcgtaccagcttcaagggcgaatccgagccgaccccagaggaacgtctgctgcgctccatcttcggcgagaaagctcgcgacgttaaggacacctccctgcgcgttccgccaggcgaaggcggtatcgttgtacgtaccgtccgtctgcgtcgtggggatccgggtgtggagctgaagccgggtgtgcgtgaggtcgtacgtgtctacgttgcccagaagcgcaagctgcaagttggcgacaagctggccaaccgccacggtaacaagggtgtggttgccaaaatcctgccggtggaggacatgccgcacctgccagacggtactccggtggacgtgatcctgaacccgctgggcgtcccaagccgtatgaacctgggtcagatcctggaaacccacctgggcctggccggttacttcctgggccagcgctacatctctccgatctttgacggtgccaaggagccggagatcaaagagctgctggctcaggcctttgaggtctacttcggcaagcgcaaaggcgaaggcttcggcgttgacaagcgtgaggtggaagtcctgcgccgtgcggaaaagctgggcctggtcaccccgggcaaaaccccggaagagcaactgaaggaactgttcctgcaaggcaaggttgtcctgtacgacggccgcacgggcgaaccgattgagggcccgatcgtcgttggtcagatgttcatcatgaagctgtaccacatggttgaggacaagatgcacgctcgctccacgggtccgtactccctgatcacccagcaaccgctgggcggtaaggcccagttcggcggtcagcgcttcggtgagatggaggtctgggcactggaggcctacggtgcggctcacaccctgcaagagatgctgaccctgaaatccgacgacattgagggccgtaacgctgcctacgaggccatcatcaagggtgaagacgttccggaaccgagcgtcccggagtccttccgcgtgctggtgaaagagctgcaagcactggccctggacgttcagaccctggacgagaaagacaacccggttgacatctttgagggtctggcctccaaacgtggccaagaaggccaaaatcaccatcaccatcaccattaatgacctaggataattctcgctcaaacaggtcactgctgtcgggttaaaacccggcagcggattgtgctaaattcgagcaccgtcgatctcgatcccgcgaaattgtagaaataattttgtttaactttaagaaggagatataccatgggcaacaaagaaattttgggtgaagatgaagccgaatccaatgaaaaggcgcaagcaggcgagaagaattacgaggccttgggtaccctggaagttctgtttcagggtccgggcgactctgcgtctggtatgaaaaaagaggttcgtaaggttcgcatcgctctggcctccccggaaaagatccgctcctggagctatggcgaggttgagaaaccggagactatcaactaccgcaccctgaagccggaacgtgacggcctgtttgacgagcgcatcttcggcccgatcaaggactacgagtgcgcctgcggcaagtacaaacgccagcgctttgagggcaaagtttgcgagcgttgcggtgttgaggtgacgaagtctatcgtccgccgctaccgcatgggtcacattgagctggccaccccggctgcccacatctggttcgtcaaggatgtcccgtccaagatcggtactctgctggacctgtccgccaccgagctggaacaggtcctgtacttcagcaagtacatcgtcctggacccgaagggtgccatcctgaacggtgtcccggtggagaaacgccagctgctgaccgacgaagagtatcgcgagctgcgctacggcaaacaggagacgtacccgctgccaccgggtgttgacgcgctggtgaaagacggtgaagaggttgtaaagggccaggagctggctccgggcgttgtcagccgcctggacggcgtggccctgtatcgcttcccgcgtcgcgttcgtgtggagtacgtgaagaaagaacgcgccggtctgcgtctgccactggctgcctgggttgagaaagaagcctacaagccgggcgaaatcctggccgagctgccggagccgtacctgttccgtgccgaggaagagggcgttgttgagctgaaagagctggaagaaggcgctttcctggtcctgcgtcgcgaggacgaacctgttgccacctacttcctgccagtgggcatgactccgctggtggtccacggtgagatcgtggaaaagggccagccactggccgaagccaaaggcctgctgcgcatgccacgccaggttcgcgccgcacaggtggaagcggaagaggaaggcgagacggtctacctgaccctgttcctggagtggacggagcctaaagactaccgcgtccagccacatatgaacgttgttgtcccggaaggtgcacgcgttgaggcgggcgacaagatcgttgccgccattgacccggaagaagaggtcatcgccgaagccgagggcgttgtccacctgcacgagccagccagcatcctggtggtcaaagcccgtgtctacccgtttgaggacgacgtggaggtttccactggcgatcgcgttgcaccgggtgatgtcctggccgacggtggcaaggtcaaaagcgacgtttacggccgcgttgaggtggatctggtccgcaacgtggtccgtgtggtggaatcctacgacattgacgcccgcatgggtgccgaggccatccagcagctgctgaaagagctggacctggaggccctggagcgcgaactgctggaagagatgaaacacccatctcgcgcacgtcgcgctaaggcccgtaaacgcctggaggttgtccgtgccttcctggactctggtaaccgtccggagtggatgatcctggaagccgtcccggtcctgccaccggacctgcgtccgatggtccaggtggacggcggtcgctttgccacgagcgacctgaacgacctgtaccgtcgcctgatcaaccgcaacaaccgtctgaaaaagctgctggcccagggcgctcctgagatcatcatccgcaacgagaaacgtatgctgcaagaagcggttgacgccctgctggacaacggtcgtcgcggcgctccagttaccaacccgggctccgaccgtccactgcgtagcctgaccgatatcctgtccggcaaacagggccgcttccgccagaacctgctgggcaagcgtgtggactactctggccgtagcgtgatcgtggttggtccgcagctgaagctgcaccagtgcggcctgccgaaacgtatggccctggaactgttcaagccgttcctgctgaagaagatggaagagaaaggcatcgccccgaacgtcaaggcggcgcgtcgtatgctggaacgccagcgtgacatcaaagacgaggtttgggacgccctggaggaagttatccacggcaaagttgtcctgctgaaccgcgccccaaccctgcaccgcctgggcatccaggccttccaaccggtcctggtggaaggtcagtccatccagctgcacccgctggtctgcgaggccttcaacgccgactttgacggtgaccagatggctgtacatgtcccactgtcctccttcgcccaggccgaagcccgcatccagatgctgtccgcccacaacctgctgtctccagcctccggtgaaccactggccaaaccgagccgtgacatcatcctgggcctgtactacatcacccaggttcgcaaagagaagaaaggcgctggtctggagttcgccaccccagaagaagccctggcagcccacgaacgcggcgaagttgccctgaacgccccgatcaaagtggcgggccgtgaaaccagcgtgggtcgtctgaagtacgtcttcgccaacccggacgaagccctgctggccgttgcccacggcatcgtggacctgcaagacgtggtcaccgtccgctacatgggcaaacgtctggagacgagcccgggtcgcatcctgttcgcccgcatcgtggccgaagcggtggaagacgagaaggttgcctgggagctgatccagctggacgttccgcaggagaagaactccctgaaagaccttgtctaccaggccttcctgcgcctgggtatggagaaaaccgcccgtctgctggacgccctgaagtactacggcttcaccttctccaccactagcggcatcaccatcggcattgacgacgccgtgatcccggaagagaaaaagcagtacctggaggaagcggaccgcaaactgctgcaaattgaacaggcctacgagatgggcttcctgaccgaccgtgagcgttacgaccagatcctgcaactgtggaccgaaaccacggagaaagtcacccaggcggtcttcaagaacttcgaagaaaactacccattcaacccgctgtacgtcatggcccagtccggtgcacgtggcaacccgcagcagatccgccagctgtgcggtctgcgtggcctgatgcagaaaccgtctggcgaaacctttgaggttccggttcgctcttccttccgcgaaggcctgaccgtcctggagtacttcatctcctctcacggtgcccgtaaaggcggtgcggataccgcactgcgtaccgccgactccggctacctgacccgcaaactggtggacgtcacccacgagatcgtggtgcgtgaagcggactgcggcaccaccaactacatctccgttccgctgttccagccagacgaagtgacccgctccctgcgcctgcgtaaacgcgcggacattgaggccggcctgtacggtcgtgtcctggcccgtgaagtggaggtcctgggtgttcgcctggaagagggtcgctacctgtctatggacgacgttcacctgctgatcaaagccgctgaagccggtgagatccaggaagttccggttcgcagcccgctgacctgccagacccgctacggtgtttgccagaagtgctacggttacgacctgtctatggcccgtccagtctccatcggcgaagcggtgggcatcgtggcagcccagtccatcggcgaaccaggcacccaattgaccatgcgtaccttccatacgggtggcgtggctggtgcagcggacatcacccagggtctcccgcgtgtcattgagctgtttgaggcccgtcgcccgaaagccaaagcggtgatctccgagattgacggtgttgttcgcattgaggaaacggaggaaaagctgtccgtcttcgttgagtccgagggcttctccaaagagtacaagctgccgaaagaggcgcgtctgctggtcaaggatggcgactacgttgaagcgggccagccactgacccgtggtgccattgacccgcatcagctgctggaagccaagggcccggaagcggttgaacgctatctggttgaagagatccagaaagtctaccgcgcccagggcgttaaactgcacgacaaacacattgagatcgttgtccgtcagatgatgaagtacgtggaagtcaccgacccgggtgacagccgcctgctggaaggccaggttctggagaaatgggatgtcgaagccctgaacgagcgtctgatcgccgaaggcaagaccccagtggcctggaaaccgctgctgatgggtgtcacgaagagcgccctgtccaccaaaagctggctgtccgcggctagcttccagaacaccacccacgtcctgaccgaagcggccatcgccggtaagaaagacgagctgatcggcctgaaggagaacgtcatcctgggccgtctgatcccggcaggtactggttctgacttcgtccgctttacccaggtggtggaccagaaaaccctgaaggccattgaggaagcccgcaaagaggcggttgaggccaaggaacgtccagccgctcgtcgcggcgtcaaacgcgaacagccgggcaaacaggctggcggccgcacttctggcaacggtgatcagggcctggaggtcctgttccagggcccgcagggtgaaaactctggcacttctggcaccgacaatggctcttccgatggcctggaagtgctgtttcaaggcccagcaggtaattctgcgtctagcggttctggtggcggtgctgaaccaggtattgataaactgttcggcatggttgattctaaataccgtctgactgttgtggtagccaaacgtgcgcagcaactgctgcgtcacggtttcaaaaacaccgttctggaaccggaagaacgccctaaaatgcagaccctggaaggcctgttcgatgatccgaatgccgtcacttgggctatgaaagaactgctgactggtcgcctggtgtttggtgaaaatctggttccggaagatcgtctgcaaaaagaaatggaacgtctgtacccggttgaacgtgaagaaactgctggcactagtgatggcaacggtgaagataaccagggcctggaagtactgtttcaaggtccgcagggtgaaaactctggctctggcgacggcgatggtggcgagtcttcttccgatggcacgctggaggtgctgttccagggtcctggcggtgactctgcacacgaaggtggcaccgcaggttccaaaagcaacgaaccgggtaaagcaactggtgagggtaaaccagttaacaacaaatggctgaacaacgccggtaaagacctgggttccccggttcctgaccgcatcgcgaataagctgcgcgataaggaatttgaatcctttgacgatttccgtgaaaccttttgggaagaagtctctaaagacccggaactgagcaaacagttctcccgcaacaacaacgaccgcatgaaagtgggcaaagctccgaaaacccgtactcaagacgtttctggcaaacgcactagctttgaactgaaccatcagaagccgattgaacaaaacggcggtgtttatgatatggacaacatctccgtggttacgccgaaacgtaacatcgatatcgaaggttaattaagctgaacaagctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 21 - DNA, 15484 bp, synthetic construct
  misc_feature 1..15484: note = Synthetic construct
  source 1..15484: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaaccttaagaaggagatataccatgctgatctctcagcgcccgaccctgtccgaagatgtcctgaccgacaaccgttcccagttcgtgatcgaaccgctggaaccgggtttcggctacaccctgggcaattctctgcgtcgcaccctgctgtcttccattccgggtgcggccgttaccagcattcgcatcgatggtgtactgcacgaatttaccacggttccgggtgtcaaagaagatgtcaccgaaatcatcctgaatctgaaaagcctggttgtatcttccgaggaagacgaaccggttaccatgtatctgcgcaaacagggtccgggtgaagttaccgccggcgacatcgtgccgccagcaggtgttaccgtgcacaacccgggcatgcacatcgccacgctgaacgataaaggcaaactggaagtcgagctggttgtcgaacgtggccgtggctatgttccggcggtgcaaaaccgcgcttccggtgccgaaattggtcgcattccagtcgattccatctactctccggttctgaaagtgacctacaaagtggacgccacccgtgtcgaacagcgtacggactttgacaaactgatcctggacgttgaaaccaaaaattctatcagcccgcgcgacgcgctggcgtccgctggcaaaacgctggtcgaactgttcggcctggcacgcgaactgaacgtcgaagccgaaggcatcgaaatcggtccatccccggccgaagccgatcacattgcgtctttcgccctgccgatcgacgatctggatctgacggttcgttcctacaactgcctgaaacgcgaaggcgttcacaccgtgggcgaactggtagcgcgcaccgaatccgacctgctggacatccgcaactttggtcagaaatccatcgacgaagtgaaaatcaaactgcaccagctgggcctgtctctgaaagacagcccgccgagcttcgacccgtccgaagtcgcgggctacgacgtagccaccggcacctggtccaccgaaggtgcgtatgacgaacaggattacgccgaaaccgaacagctgtaatgaatttaaatagtaaatatggcctcaaataacgtaaaaacccgcttcggcgggtttttttatggggggagtttagggaaagagcatttgtcagaatcaagcatcaatgactgcagacttaagaaggagatataccatgagcgataaaattaatcaccagactgacgacagttatgacacggatgtagacaaagcggacggcgcgaaccaggtcgactactccgcagagctggaagtcctgttccaaggccctggtcaagcttctggtgcggacggtggtatgctggaaggttgcatcctggcagattcccgccagagcaaaactgccgcttctccttctccgtctcgcccgcaatcttccagcaataactccgtaccgggcgcgccaaaccgtgtctccttcgctaaactgcgcgaaccactggaagttccgggtctgctggatgttcagaccgattcctttgaatggctgatcggttctccgcgctggcgcgaatccgcagccgaacgtggtgatgtcaacccagtgggtggcctggaagaagtgctgtacgaactgtctccgatcgaggacttctccggttctatgtctctgtccttctctgaccctcgtttcgacgatgtcaaagcaccagtagacgagtgcaaagacaaagacatgacgtatgcggctccactgtttgtcaccgccgagttcatcaacaacaacaccggtgaaatcaaatctcagacggtgttcatgggtgacttcccgatgatgaccgagaaaggcacgttcatcatcaacggtactgaacgtgtagttgtcagccagctggtgcgttctccgggtgtgtacttcgacgaaaccattgacaaatccaccgacaaaacgctgcatagcgtcaaagtgatcccgagccgcggtgcgtggctggagtttgatgtcgataaacgcgacaccgtcggcgtgcgcatcgaccgcaaacgccgtcaaccggtcaccgttctgctgaaagcgctgggttggaccagcgaacagattgtcgaacgtttcggtttctccgagatcatgcgttctacgctggagaaagacaacactgtaggcaccgatgaagcgctgctggacatctaccgcaaactgcgtccgggcgaaccaccgaccaaagaatctgcgcagacgctgctggaaaacctgttcttcaaagagaaacgctacgacctggcccgcgtcggtcgctataaggtcaacaaaaaactgggtctgcatgtcggcgagcctatcacgtcttctacgctgaccgaagaggatgtcgtggccaccatcgaatatctggtccgcctgcacgaaggtcagaccacgatgaccgttccgggtggcgtcgaagttccggttgaaaccgatgacattgaccacttcggcaaccgtcgcctgcgtactgtcggcgaactgatccaaaaccagatccgtgtcggcatgtctcgtatggagcgtgttgtccgtgagcgtatgaccacccaggatgttgaagcgatcactccgcaaactctgatcaacattcgtccggtggtcgccgcgatcaaagaattcttcggcacctctcagctgagccaatttatggatcagaacaacccgctgtctggtctgacccacaaacgtcgcctgtctgcgctgggtccaggcggtctgtctcgtgagcgtgccggtctggaagtccgcgacgttcacccgtctcattacggccgtatgtgcccgatcgaaacccctgaaggtccaaacatcggtctgatcggctctctgtctgtttacgcgcgtgtcaacccgttcggtttcatcgagaccccgtaccgcaaagttgttgacggcgtagttagcgacgaaatcgtgtacctgaccgcagacgaagaagaccgccacgtcgttgcacaggccaattctccaatcgatgcggacggtcgcttcgtcgaaccgcgcgttctggtccgtcgcaaagcgggcgaagttgaatacgtgccgtcttctgaagtagactacatggacgtttctccacgccagatggtgtccgtggcaaccgctatgattccgtttctggaacacgatgacgccaaccgtgccctgatgggtgcaaacatgcagcgccaggcggttccgctggtccgtagcgaagccccgctggttggcaccggtatggaactgcgcgcagcgatcgacgcaggcgatgtcgttgtagccgaagaaagcggcgtcatcgaagaagtgtctgccgactacatcactgtgatgcacgataacggcacccgtcgtacctaccgtatgcgcaagtttgcccgttccaaccacggcacttgcgccaaccagtgcccaatcgtggacgcgggtgaccgtgttgaggccggccaggttatcgccgatggtccatgtactgacgacggcgaaatggcgctgggcaaaaacctgctggttgccatcatgccgtgggaaggccacaactacgaagacgcgatcattctgtccaaccgcctggtcgaagaagacgttctgacctctatccacatcgaagaacatgagatcgatgctcgcgacaccaaactgggtgcggaagagatcacccgcgacattccgaacatctccgacgaagttctggccgacctggatgaacgtggcattgtacgtattggtgccgaagttcgcgacggtgacatcctggtcggcaaagtcaccccgaaaggtgagactgaactgacgccggaagaacgtctgctgcgtgccatcttcggtgagaaagcacgcgaagtgcgcgacacttctctgaaagtgccgcacggcgaatccggcaaagttatcggcattcgtgttttttcccgcgaagacgaagatgagctgcctgcaggtgtcaatgaactggttcgtgtgtatgtagctcagaaacgcaaaatctccgatggtgataaactggctggccgtcacggcaacaaaggcgtaatcggcaaaatcctgccggttgaagacatgccgttcctggccgacggcaccccggttgacattattctgaacacccacggcgtaccgcgtcgtatgaacatcggccagattctggaaacccacctgggttggtgtgcccacagcggctggaaagtcgatgcagccaaaggtgttccggactgggctgcccgtctgccagacgaactgctggaagcgcaaccgaacgccattgtgtctacgccggtattcgacggtgcccaggaagccgaactgcagggcctgctgtcttgcacgctgcctaaccgcgatggtgacgttctggtagatgccgacggcaaagccatgctgttcgacggtcgcagcggcgaaccgttcccgtacccggtcacggttggctacatgtatatcatgaaactgcaccatctggtggacgacaaaatccacgcccgctccaccggtccgtactctatgatcacccagcagccgctgggcggtaaagcgcagttcggtggccagcgtttcggtgaaatggaatgctgggcaatgcaggcatacggtgctgcctacactctgcaggaactgctgaccatcaaatccgatgacactgttggccgcgttaaagtgtacgaagcgatcgtcaaaggtgaaaacatcccggaaccgggcatcccggaatctttcaaagtgctgctgaaagaactgcagagcctgtgcctgaacgtcgaagtgctgtcctctgacggtgcggcgatcgaactgcgtgaaggtgaagatgaagacctggaacgtgccgcagccaacctgggtatcaatctgtcccgcaacgaatccgcatctgtcgaagatctggcgggccaagaaggccaaaatcaccatcaccatcaccattaatgacctaggataattctcgctcaaacaggtcactgctgtcgggttaaaacccggcagcggattgtgctaaattcgagcaccgtcgatctcgatcccgcgaaattgtagaaataattttgtttaactttaagaaggagatataccatgggcaacaaagaaattttgggtgaagatgaagccgaatccaatgaaaaggcgcaagcaggcgagaagaattacgaggccttgggtaccctggaagttctgtttcagggtccgggcgactctgcgtctggtatgctggatgtcaacttctttgatgaactgcgtatcggtctggctaccgcagaagacatccgtcaatggtcctatggcgaagtcaaaaaaccggaaacgatcaactatcgcacgctgaaaccggagaaagacggcctgttctgcgagaaaatcttcggtccaactcgcgattgggaatgctactgcggcaaatacaaacgtgtgcgcttcaaaggcatcatctgcgaacgctgcggcgtcgaagttacccgcgccaaagttcgtcgtgaacgtatgggccacatcgaactggccgcgccagtcacccatatctggtacttcaaaggtgtgccttctcgtctgggttatctgctggacctggccccgaaagacctggagaaaatcatctacttcgctgcatatgttatcacctctgtagacgaagaaatgcgccacaatgaactgtccacgctggaagccgaaatggcggttgaacgcaaagccgtcgaagaccagcgcgacggcgaactggaagcccgtgcgcaaaaactggaagccgacctggccgagctggaagcagaaggcgccaaagccgatgcgcgtcgcaaagttcgcgacggtggcgaacgcgaaatgcgccagatccgtgaccgcgcacagcgtgaactggaccgtctggaagacatctggagcactttcaccaaactggcgccaaaacagctgatcgttgacgaaaacctgtatcgcgaactggtcgatcgctacggcgaatacttcaccggtgccatgggcgcggaatctatccagaaactgatcgaaaacttcgacatcgacgccgaagccgaatctctgcgtgatgtcatccgtaacggcaaaggtcagaaaaaactgcgcgccctgaaacgtctgaaagtggttgctgcgttccaacagtctggcaactctccgatgggcatggttctggacgcagtcccggttatcccaccggagctccgcccgatggttcagctggacggtggccgtttcgccacgtccgacctgaacgacctgtaccgccgtgtaatcaaccgcaacaatcgtctgaagcgtctgatcgatctgggtgctccggaaatcattgtcaacaacgaaaaacgtatgctgcaggaatccgttgacgcgctgttcgacaatggccgtcgcggtcgtcctgtcactggtccgggcaaccgtccgctgaaatctctgtccgatctgctgaaaggcaaacagggccgtttccgtcagaacctgctgggcaaacgtgtcgattactctggccgtagcgtcatcgtagtcggccctcagctgaaactgcatcagtgcggtctgcctaaactgatggcgctggaactgttcaaaccgttcgttatgaaacgtctggtagacctgaaccatgcgcagaacatcaaaagcgccaaacgcatggttgaacgccagcgccctcaagtgtgggatgtgctggaagaagtcatcgccgagcacccggtgctgctgaaccgcgcaccaaccctgcaccgtctgggtatccaggcgttcgaaccaatgctggttgaaggcaaagccattcagctgcacccgctggtatgtgaagcgttcaatgccgacttcgatggtgaccagatggctgtacacctgcctctgagcgccgaagcgcaggccgaagctcgcattctgatgctgtcttccaacaacatcctgtctccggcatctggtcgtccgctggccatgccgcgtctggacatggtgaccggtctgtactatctgaccaccgaggtccctggtgacaccggcgaataccagccagccagcggtgatcacccggaaactggtgtctactcttctccggccgaagcgatcatggcagccgaccgtggtgttctgagcgtgcgtgccaaaatcaaagtgcgtctgacccagctgcgtccaccggtcgaaatcgaagccgaactgtttggccacagcggctggcagccgggcgatgcgtggatggccgaaaccacgctgggccgtgtgatgttcaacgaactgctgccgctgggttatccgttcgtcaacaaacagatgcacaagaaagtgcaggctgccatcatcaacgacctggccgaacgttacccgatgatcgttgtcgcccagaccgtcgataaactgaaagatgccggcttctattgggccacccgcagcggcgtaacggtttctatggccgacgttctggttcctcctcgcaaaaaggaaatcctggaccactacgaagaacgcgcggacaaagtcgaaaagcagtttcagcgtggcgctctgaaccacgacgaacgcaacgaagcgctggtagaaatttggaaagaagccaccgacgaagtcggtcaggcgctgcgtgaacactacccagacgataacccgatcattaccattgtcgattccggtgccaccggcaacttcacccagactcgtactctggcaggtatgaaaggtctggtaaccaacccgaaaggtgagttcatcccgcgtccggtcaaatcctccttccgtgaaggcctgaccgttctggaatacttcatcaacacccatggcgctcgtaaaggtctggcggacaccgcgctgcgcaccgccgattccggctacctgacccgtcgcctggtagacgtctcccaggacgttatcgttcgcgaacacgattgccagaccgaacgcggcatcgtagtcgaactggctgaacgtgcaccagacggcacgctgatccgcgacccgtacatcgaaacctctgcctacgcgcgtaccctgggcaccgacgcagtcgatgaggctggcaacgtcatcgtcgaacgtggtcaagacctgggcgatccggaaattgacgctctgctggctgctggtattacccaggtcaaagtgcgttctgtactgacgtgtgccaccagcaccggcgtttgcgcgacctgctacggtcgttccatggccaccggcaaactggtcgatatcggtgaagccgtcggcatcgtagccgctcagtccatcggcgaacctggcacccaattgaccatgcgcacctttcatcagggtggcgtcggtgaagacatcaccggtggtctgccacgtgttcaggaactgtttgaagcccgtgttccgcgtggcaaagcgccgatcgccgatgtcaccggccgtgttcgtctggaagacggcgaacgtttctacaaaatcaccatcgttcctgacgatggcggtgaagaagttgtctacgacaaaatctccaaacgtcagcgtctgcgtgtgttcaaacacgaagacggttccgaacgtgtgctgtccgatggcgaccacgtcgaggtaggccagcagctgatggaaggcagcgcggatccacatgaagtgctgcgtgttaaaggcccacgcgaagtacagattcacctggttcgcgaagtccaggaagtctaccgcgcccaaggtgtttctatccacgacaaacacatcgaagtaatcgttcgccagatgctgcgccgtgttaccatcattgactctggctctacggagtttctgcctggcagcctgatcgatcgcgcggaatttgaagcagaaaaccgccgtgtagttgccgaaggcggtgaacctgcggccggtcgtccggtactgatgggcatcacgaaagcgtctctggccaccgactcttggctgtctgcggctagctttcaggaaaccactcgcgttctgaccgatgctgcgatcaactgccgcagcgacaaactgaacggtctgaaagaaaacgtgatcatcggcaaactgatcccggccggtactggtatcaaccgctatcgcaacatcgcggtacagcctaccgaagaagcccgcgctgcagcgtacaccatcccgtcttatgaagatcagtactacagcccggactttggtgcagccaccggtgctgccgtcccgctggatgactacggctacagcgactatcgtggcggccgcactggttctggcaacggtgaagataaccagggcctggaggtcctgttccagggcccgcagggtgaaaactctggctctggcgacggcgatggtggcgagtcttcttccgatggcacgctggaagtgctgtttcaaggcccaactggtaattctgcgtctagcggtgatgaacagaacgcaggttctggtggcggtatgtctatcagccagtccgatgcgagcctggcagcagtcccagccgttgatcagttcgatccgtcttccggtgcatctggtggctatgacaccccgctgggcattaccaatccgcctattgacgaactgctggaccgcgtttctagcaaatacgccctggtgatctatgcggcaaaacgtgcccgtcagatcaacgattactacaaccagctgggcgaaggcatcctggaatatgttggtccgctggttgaaccgggtctgcaagaaaaaccgctgtccatcgcgctgcgcgagatccacgccgatctgctggaacacaccgaaggcgaaggtactgctggcactagtgatggcaacggtgaagataaccagggcctggaagtactgtttcaaggtccgcagggtgaaaactctggctctggcgacggcgatggtggcgagtcttcttccgatggcacgctggaggtgctgttccagggtcctggcggtgactctgcacacgaaggtggcaccgcaggttccaaaagcaacgaaccgggtaaagcaactggtgagggtaaaccagttaacaacaaatggctgaacaacgccggtaaagacctgggttccccggttcctgaccgcatcgcgaataagctgcgcgataaggaatttgaatcctttgacgatttccgtgaaaccttttgggaagaagtctctaaagacccggaactgagcaaacagttctcccgcaacaacaacgaccgcatgaaagtgggcaaagctccgaaaacccgtactcaagacgtttctggcaaacgcactagctttgaactgaaccatcagaagccgattgaacaaaacggcggtgtttatgatatggacaacatctccgtggttacgccgaaacgtaacatcgatatcgaaggttaattaagctgaacaagctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 22 - protein, 8 aa, synthetic construct
  REGION 1..8: note = Synthetic construct
LEVLFQGP

SEQ ID NO: 23 - skipped

(19) United States
(12) Patent Application Publication
(10) Pub. No.: US 2024/0417426 A1
(43) Pub. Date: Dec. 19, 2024

(54) BACTERIAL COLICIN-IMMUNITY PROTEIN PROTEIN PURIFICATION SYSTEM

(71) Applicant: THE UAB RESEARCH FOUNDATION, Birmingham, AL (US)

(72) Inventors: Dmitry Vassylyev, Vestavia Hills, AL (US); Norman Patrick Higgins, Birmingham, AL (US); Marina Vassylyeva, Vestavia Hills, AL (US); Alexey Vasiliev, Vestavia Hills, AL (US)

(73) Assignee: THE UAB RESEARCH FOUNDATION, Birmingham, AL (US)

(21) Appl. No.: 18/672,712

(22) Filed: May 23, 2024

Related U.S. Application Data
(63) Continuation of application No. 16/938,377, filed on Jul. 24, 2020, now Pat. No. 12,030,913.
Application No. 16/938,377 is a division of application No. 16/060,753, filed on Jun. 8, 2018, now Pat. No. 10,759,830.
(60) Provisional application No. 62/265,253, filed on Dec. 9, 2015.

(51) Int. Cl. C07K 1/22; C07K 14/245; C12N 9/22; C12N 15/62; C12P 21/02; C12P 21/06

(52) U.S. Cl. CPC C07K 1/22; C07K 14/245; C12N 9/22; C12N 15/62; C12P 21/02; C12P 21/06; C07K 2319/00; C07K 2319/21; C07K 2319/23; C07K 2319/24; C07K 2319/50; C12N 2310/20

(57) ABSTRACT

Provided herein are compositions and methods for protein purification.

This application is a continuation of U.S. application Ser. No. 16/938,377, filed on Jul. 24, 2020, which is a divisional of U.S. application Ser. No. 16/060,753, filed on Jun. 8, 2018, now issued U.S. Pat. No. 10,759,830, which is a U.S. national stage application under 35 USC § 371 of PCT/US2016/065843, filed on Dec. 9, 2016, which claims the benefit of U.S. Provisional Application No. 62/265,253, filed Dec. 9, 2015, which are incorporated by reference herein in their entirety.

### SEQUENCE LISTING

The instant application contains a Sequence Listing in XML format. The Sequence Listing, named UAB-179US3_ST26_035979-1440890.xml, which was created on May 2, 2024, is 100 KB in size, and is hereby incorporated by reference in its entirety.

### BACKGROUND

Most commercially available purification tools are not useful for complex biological systems. Thus, protein purification can be an unpredictable, multi-step process. Protein purification of multi-subunit complexes and membrane proteins is particularly challenging as currently available approaches are time-consuming and fail to consistently provide samples of high quality, high yield and high purity (HHH).

### SUMMARY

The present disclosure relates to purification of proteins, including multi-subunit complexes and membrane proteins. Provided herein are non-naturally occurring polypeptides and methods of using one or more of the polypeptides in purification methods. Also provided are related nucleic acids, vectors, genetically modified cells and affinity matrices.

More specifically, provided herein are polypeptides comprising a wild-type colicin-DNAse domain modified to comprise one or more mutations selected from the group consisting of a mutation that reduces DNAse activity, a mutation that decreases DNA binding and a mutation that increases thermostability of the polypeptide. The polypeptides optionally comprises a heterologous polypeptide that is operably linked to a cleavable polypeptide sequence, wherein the cleavable polypeptide sequence links the heterologous polypeptide with the colicin-DNAse domain.

Further provided are polypeptides comprising a colicin immunity protein, wherein the immunity protein comprises one or mutations that increase thermostability of the polypeptide.

The methods provided herein include a) transfecting in a cell culture medium a cell with a vector, wherein the vector comprises a nucleic acid encoding a first polypeptide under conditions in which the first polypeptide is expressed, wherein the first polypeptide is a polypeptide comprising a heterologous protein and a wild-type colicin-DNAse domain modified to comprise one or more mutations, wherein the heterologous protein and the modified colicin-DNAse domain are linked by a cleavable polypeptide sequence; b) harvesting the cell culture medium comprising the expressed first polypeptide; c) lysing the cells to obtain a supernatant comprising the expressed first polypeptide; d) contacting the supernatant with an affinity matrix comprising a substrate and a second polypeptide, wherein the second polypeptide comprises a colicin immunity protein with one or more mutations, e) washing the matrix to remove biological molecules non-specifically bound to the first expressed polypeptide and the matrix; and f) eluting the heterologous protein from the matrix, comprising enzymatically cleaving the heterologous protein from the first polypeptide.

Also provided are methods that include a) transfecting in cell culture medium a cell with a vector comprising a nucleic acid encoding a first polypeptide under conditions in which the first polypeptide is expressed, wherein the first polypeptide is a polypeptide comprising a heterologous protein and a wild-type colicin-DNAse domain modified to comprise one or more mutations, wherein the heterologous protein and the modified colicin-DNAse domain are linked by a cleavable polypeptide sequence; b) harvesting the cell culture medium comprising the expressed first polypeptide comprising the heterologous protein; c) contacting the harvested cell culture medium with an affinity matrix, wherein the affinity matrix comprises a substrate and a second polypeptide comprising a colicin immunity protein with one or more mutations; d) washing the matrix to remove biological molecules non-specifically bound to the expressed first polypeptide and the matrix; and e) eluting the heterologous protein from the matrix, comprising enzymatically cleaving the heterologous protein from the first polypeptide.

### DESCRIPTION OF THE DRAWINGS

FIG. 1A shows the expression and purification of an immunity protein 7 (Im7 (LG)) that can be immobilized on a substrate and used in a single column protein purification system.

FIG. 1B shows a schematic of purification of tagged proteins using an Im7 column.

FIG. 1C shows the expression and purification a 45 kDa protein (Trx→RC→SUMO). RC-LG (Im7) receptor, CL7-tag.

FIG. 2A shows the expression and purification of a multi-subunit RNA polymerase from T. thermophilus.

FIG. 2B shows the expression and purification of a multi-subunit RNA polymerase from M. tuberculosis. Each of these proteins consists of five protein subunits (α₂,β,β′,ω) with the total MW of ˜400 kDa.

FIG. 3A shows the expression and purification of Yidc, a transmembrane bacterial integrase.

FIG. 3B shows the the expression and purification of calnexin, a transmembrane human chaperone protein.

FIG. 4 is a schematic of the vectors constructed for protein expression and purification using a CL7/Im7 (RC/LG) affinity system.

FIG. 5A shows the structural similarity of bacterial (E. coli) and mammalian (human) condensins (condensin I and condensin II). The genetic structure of the E. coli and Salmonella Typhimurium DNA condensin (or MukFEB operons) is also shown. Transcription initiates near the smtA gene, which encodes an S-adenosyl methionine transferase. All four genes of this operon are translationally coupled to the previous gene. A ribosome translates every protein without dissociating from mRNA. The One Column tag was introduced by inserting a module precisely after the last amino acid of MukB creating a MukB-Tag fusion. Selecting for kanamycin resistance facilitates this genetic method.

FIG. 5B shows the expected structure of a MukFEB condensin was two MukB and MukE subunits with only one copy of MukF. FIG. 5C shows the results of condensin purification using the One Column approach. Four grams of protein dissolved in 200 ml of buffer was applied to a 1.5 ml One Column. After extensive washing with 100 column volumes of high and then low salt buffer, all proteins were released from the column by applying 6 M Guanidine H—Cl, yielding 750 μg of a highly purified fraction.

FIG. 5C shows an SDS gradient gel stained with Coomassie Blue. The M lane contains highly purified Biorad protein markers. Bands in the product lane (P) were excised and identified by mass spectroscopy. MukF, E, and B proteins were present in ratios that agree with the model on the left. However, the percentage of protein released from a second column in which 90% of the tags were cleaved at a Precision Protease cleavage site showed that, in this case, each complex has approximately 10 tags and all must be cleaved to release protein from the column.

FIG. 5D shows a MukB octamer structure.

FIG. 6A shows the performance of most widely used purification techniques. Comparative results with the CL7/Im7 system are shown. Abbreviations are as follows: CalBD—calmodulin binding domain; MBP—maltose binding protein; GST—gluthathione-S transferase; ChBD—chitin binding domain; CL7/Im7—colicin E7 DNAse domain and its inhibitor, Immunity protein 7; Prot—-protein; Pept—short peptide; SM—small molecule; PP—(Price/Performance)=(Price of 1 ml beads)/(Amount of protein bound to 1 ml beads); EL—eluate.

FIG. 6B shows the results of using a one-step His-Tag purification of multi-subunit (α₂ββ′ω) DNA-binding M. tuberculosis RNA polymerase (mtRNAP; left), bacterial transmembrane integrase, Yidc (middle), and two human kinases expressed in host cells.

FIG. 7A shows the wild-type CL7-DNAse amino acid sequence (SEQ ID NO: 1). Active site residues (H99, H124 and H128 of SEQ ID NO: 1) and DNA-binding residues (R2, K4, K11, K45, K51, K52, S105 of SEQ ID NO: 1) that were modified as set forth herein are highlighted.

FIG. 7B shows a schematic protocol for purification of CL7-tagged target proteins using the Im7 protein coupled to Sulfo-Link 6B agarose beads, and expression and purification of purification a 45 kDa protein (Trx→RC→SUMO). Abbreviations are as follows: SUMO, SUMO-P—-SUMO domain and SUMO protease, respectively; P(PSC), PSC—prescission protease binding site and protease itself, respectively; T—target protein; G-HCl—-guanidine hydrochloride; H8—8 histidine tag; Trx—thioredoxine; WCL—whole cell lysate; FT—flow through; EL—eluate.

FIG. 7C shows expression and purification of an Im7 immobilization unit.

FIG. 7D shows expression and purification of a CL7-tagged protein.

FIG. 8A show examples of multi-subunit RNAP expression vectors. FIG. 8B is a gel showing expression levels of ttRNAP using the purification systems described herein. FIG. 8C is a gel showing one-step purification of ttRNAP using the His-tag (left panel) and (CL7/Im7) (right panel) approaches. FIG. 8D shows the results of transcription elongation assays for overexpressed (left panel) and expressed (right panel) ttRNAP from host thermophilic cells. Abbreviations are as follows: T7P—T7 promoter; RB—ribosme stating site; ST—stop codon; EX—the short N-terminal “expression” peptides from the maltose-binidng protein (EX1, 25 a.a.), thioredoxin (EX2, 30 a.a.), and NusA (EX3, 30 a.a); H8—8 histidine tag; P(PSC)—prescission protease cleavage site; PSC—prescission protease; WCL—whole cell lysate; WCL1—lysate (WCL) heated at 70 C for ˜45 mins; FT—flow through; EL—eluate; RNA18—synthetic 18-mer RNA olig labeled with fluoresceine (FLU) at the 5′-end. T/NT—DNA template (T) and non-template (NT) strands in the synthetic transcription elongation scaffold.

FIG. 9A shows the results of one-step purification of mtRNAP using the His-tag (left panel) and (CL7/Im7) (right panel) approaches. FIG. 9B shows transcription elongation assays for overexpressed mtRNAP (left panel) and commercial E. coli RNAP (ecRNAP, right panel). Abbreviations are as follows: PSC—prescission protease; WCL—whole cell lysate; FT—flow through; EL—eluate; RNA18—synthetic 18-mer RNA olig labeled with fluoresceine (FLU) at the 5′-end. T/NT—DNA template (T) and non-template (NT) strands in the synthetic transcription elongation scaffold.

FIG. 10A shows the results of expression and a one-step purification of Yidc using the His-tag approach and the wild-type (WT, not adjusted for the E. coli codons) sequence. FIG. 10B shows the results of expression (with IPTG induction) of Yidc with the gene sequence adjusted for E. coli codons and C-terminal C17 tag. With the enhanced expression, Yidc and the other membrane proteins remain in the soluble fraction (SN) upon ultracentrifugation (left panel), but precipitates with DNA upon polyethylenimine (PE) precipitation in ˜0.3-0.35M NaCl (right panel). FIG. 10C shows the results of a one-step purification of Yidc using the (CL7/Im7) approach. Purification using ultracentrifugation of the non-induced cells and the WT signal peptide (SP) sequence, with Cys residue at the N-terminus of the Yidc after cleavage by the signal peptidase is shown. FIG. 10D shows the results of a one-step purification of Yidc using the (CL7/Im7) approach. Purification with no ultra-centrifuge step but using the PE precipitation instead and with the N-terminal Cys/Ser mutation is shown. Abbreviations are as follows: H6—6 histidine tag; PSC—prescission protease; P(TEV)/P(PSC)—TEV/PSC cleavage sites; MF—membrane fraction (ultra-centrifuge pellet dissolved in 1.5% dodecyl-maltopyranoside, DDM); Wash—cloumn wash with 30/50 mM imidazole; WCL—whole cell lysate; SN—supernatant (soluble fractions); PE(PL)—supernatant after PE pellet is washed with the buffer containing 0.6M NaCl and 1.5% DDM; Cells (3/20 hrs)—uninduced (no IPTG) cells after 3/20 hrs proliferation; FT—flow through; EL—eluate; SP_{WT}, SP0_{EC}, SP1_{EC}—Yidc signal peptide with the WT (SP_{WT}), adjusted for E. coli codons (SP0_{EC}) gene sequences, and with E. coli adjusted sequence containing the (C/S) mutation.

FIG. 11 shows the results of expression and a one-step (CL7/Im7) purification of human chaperone, calnexin (CNX). Abbreviations are as follows: PSC—prescission protease; P(PSC)—PSC cleavage sites; WCL—whole cell lysate; PE—polyethyleniimine; SN/PL—supernatant (SN) and washed (0.6M NaCl; 1.5% DDM); pellet after PE precipitation; FT—flow through; EL—eluate; ΔCNX—truncation of the CNX protein; SP1_{EC}—Yidc signal peptide with the adjusted for E. coli codons gene sequence, and containing the (C/S) mutation at the N-terminal position of a protein cleaved by signal peptidase.

### DETAILED DESCRIPTION

Provided herein are compositions and purification methods for purifying any protein of interest, including multi-subunit complexes and membrane proteins, with high quality, high yield and high purity.

Protein purification is an essential, primary step in numerous studies, including proteomics and structural genomics. Most of these studies require three dimensional (3D) structures of the biological targets of interest, which can provide mechanistic insights into their functional properties. Given that obtaining a 3D structure often requires large quantities of highly purified proteins, purification tools that conform to the high yield, high purity and high activity rule (HHH rule) are necessary. Recently, studies of huge multi-subunit complexes (MSC) (transcription and translation machineries, for example) and membrane proteins (MPs) have emerged as a major focus of proteomics and its structural counterpart. Most commercially available purification tools are based on relatively small, monomeric proteins, and are not always useful for complex biological systems. Thus, protein purification has remained an unpredictable, time consuming, multi-step process, rather than a routine technical task. MSCs and MPs are particularly challenging as the purification processes usually take more time, compared to functional and/or structural studies, and often cannot provide HHH-grade samples.

At present, there are no commercially available affinity systems that completely satisfy the HHH-rule. Most commercial columns provide modest yields of only a few milligrams due to a number of factors, including the use of a small amount of antibody, chitin, biotin, etc.; poor loading or slow binding; and high kₒₙ affinity (maltose binding protein (MBP) or glutathione synthase (GST)) of the active groups. The latter (MBP and GST) are also sensitive to high salt (over 200 mM NaCl) during loading, which can affect the final purity of the target. High salt particularly affects the nucleic acid (NA) binding protein complexes, which constitute a large pool of the biologically and industrially significant systems. In fact, the His-Trap (Ni²⁺⁻based) approach is the only affinity approach with the H-yield capacity, and, for this reason, is the most popular purification technique among researchers in the field as well as in commercial applications.

However, this approach has a number of limitations that can affect the H-purity/H-activity components of the HHH-rule. For example, His-Trap columns are sensitive to reducing agents (β-ME, DTT, over 2-4 mM) and metal chelating agents (EDTA, over 1 mM). Further, column affinity is target-dependent, which requires adjustments of conditions for each new project. There can also be relatively high, non-specific affinity to DNA and cellular proteins, including many MPs. In addition, excessive amounts of Ni²⁺ ions can affect conformation and the activity of a target protein. These restrictions typically result in only 60-80% purity for the over-expressed NA-binding MSCS (for example, a multi-subunit RNA polymerase) and 50-70% purity for the MP in a one-step purification through His-Trap columns. Consistently, the His-tag labelled naturally expressed proteins are substantially less pure due to a very poor signal-to-noise ratio. Since most of the other chromatography techniques are even less specific than His-trap, i.e. more target-dependent, the final H-purity is achieved through a combination of sequential purification steps using various commercial columns specific to each particular protein. Columns using anion/cation exchange, gel-filtration, heparin, DNA-agarose, etc. necessarily increase the time required for the entire process. Costs are also increased, as researchers must purchase and maintain a number of commercial chromatography systems, some of which can cost thousands of dollars. In summary, no chromatography system currently exists that allows for predictable and efficient one-step or multi-tag purification of complex MSC or MP targets using overexpression or natural expression protein preparation protocols.

The systems provided herein overcome the challenges of existing commercial products. Using the systems and methods described herein about 97-100% purity of most intact (untruncated) targets was obtained in a one-step purification. Importantly, large scale HHH-purifications of a number of the most challenging and biologically significant MSCs and MPs was successfully performed. The purification systems provided herein offer several advantages that provide significant improvements over commercially available chromatography systems. For example, these systems are not sensitive to high salt (tested up to 1.5M NaCl), metal-chelating and reducing agents, (for example, EDTA and β-mercaptoethanol, up to 20 mM tested) or detergents. Since detergents are unavoidable during purification of MPs, lack of sensitivity of detergents ensures binding affinity and purity. These systems are also fast binding systems, which allows for flow rates of about 5 ml/min. No loss in binding capacity was observed at these flow rates, which is essential for efficient purification of proteins from natural expression systems, where large lysate volumes are often used to achieve high yield. Ultra-high affinity was achieved with receptor/ligand (RC/LG) complexes (for example, CL7/Im7 complexes) that dissociate only in 6M GuHCl. Tests showed no detectable non-specific binding to all untagged cellular molecules. High affinity is crucial for successful purification of naturally expressed proteins, as they all possess poor signal-to noise-ratio during purification. These systems are also high capacity systems that can be used to purify proteins of up to 80 kDa in size, in quantities of 400 mg or more using the 20 ml column. These systems can also employ multiple RC/LG complexes.

There are at least four known homologous (CL/Im) systems (see below), which, in spite of overall high structural and sequence similarity, possess essentially distinct binding sites and, therefore, demonstrate big losses in binding affinities (6 to 9 orders of magnitude in Kₘ, in particular, with k_{off} approaching to 0) towards the non-cognate partners. This property of the (CL/Im) complexes allows for construction of at least four original, cross-resistant chromatography systems, in which distinct, modified CL DNAse domains may be used for tagging different subunits of MSC. The multi-tag approach can be used to avoid impurities related to possible translational/proteolytic truncations and/or to account for an imbalance in the expression levels of the individual subunits, which often occurs if the MSC is overexpressed, in particular, in a foreign cell or organism.

### Polypeptides Comprising a Modified Colicin-DNAse Domain

Provided herein are polypeptides comprising a wild-type colicin-DNAse domain modified to comprise one or more mutations. The mutations are selected from the group consisting of a mutation that reduces DNAse activity, a mutation that decreases DNA binding and a mutation that increases thermostability of the polypeptide. Optionally, one or more mutations decrease DNAse activity, one or more mutations decrease DNA binding and/or one or more mutations increase thermostability. In any of the polypeptides provided herein, the wild-type colicin DNAse domain can be a wild-type colicin DNAse domain from any Escherichia coli colicin, including but not limited to, a wild-type colicin DNAse domain from colicin E7 (CL7), colicin E2 (CL2), colicin E7 (CL7) or colicin E9 (CL9). For example, the wild-type DNAse domain can be amino acids 446-573 of CL7, as set forth under GenBank Accession No. YP_009060493.1. A wild-type DNAse domain can also be an amino acid sequence from colicin colicin E2 (CL2) (Genbank Accession No. YP_002221664.1), colicin E8 (CL8) (GenBank Accession No. YP_002993419.1) or colicin E9 (CL9) GenBank Accession No. YP_002533537.1) that corresponds to amino acids 446-573 of GenBank Accession No. YP_009060493.1. The wild-type DNAse domain can also be a wild-type DNAse domain comprising an N-terminal truncation of one, two, three, four or five amino acids.

A wild-type colicin DNAse domain binds to DNA and possesses DNAse enzymatic activity. Therefore, a mutation that reduces DNA binding activity and/or DNAse enzymatic activity is a mutation that reduces DNA binding activity and/or enzymatic activity of the wild-type colicin DNAse domain. The mutation can also reduce DNA binding activity and/or DNAse enzymatic activity of the polypeptide comprising the modified DNAse, domain, as compared to a polypeptide comprising a wild-type DNAse domain. Optionally, in the polypeptides described throughout, one or more mutations are non-naturally occurring mutations.

The reduction or decrease in DNA binding activity refers to complete elimination or partial reduction. Thus, the reduction can be about a 10%, 20%, 30%, 40%, 50%, 60%, 70%, 80%, 90%, 100% reduction, or any percent reduction in between 10% and 100%, in DNA binding activity as compared to the DNA binding activity of the wild-type colicin DNAse domain. Similarly, the reduction or decrease in DNAse enzymatic activity can be partial or complete, including, for example, a 10%, 20%, 30%, 40%, 50%, 60%, 70%, 80%, 90%, 100%, or any percent reduction in between 10% and 100% reduction, in DNAse enzymatic activity as compared to the wild-type colicin DNAse domain. For example, and not to be limiting, the reduction or decrease in DNA binding activity can be a reduction of at least 10% DNA binding affinity and the reduction in enzymatic activity can be at least a 50% reduction in enzymatic activity.

The wild-type colicin-DNAse domain can be modified to comprise one or more mutations that increase the thermostability of the polypeptide as compared to the thermostability of the polypeptide in the absence of the modification, i.e., one or more mutations, in the wild-type DNAse domain. Optionally, for any of the polypeptides described throughout, an increase in thermostability results in a polypeptide that is stable at temperatures of about 60° C. or greater. For example, the polypeptide is stable at about 60° C., 70° C., 80° C. or greater, including, for example about 60-80° C.

In the polypeptides provided herein, a single mutation or a set of mutations can change one or more properties of the polypeptide simultaneously. For example, a mutation or set of mutations can reduce DNA binding and DNAse activity, a mutation or set of mutations can reduce DNA binding and increase thermostability of the polypeptide, a mutation or set of mutations can reduce DNAse enzymatic activity and increase thermostability of the polypeptide, or a mutation or set of mutations can reduce DNAse enzymatic activity, reduce DNA binding and increase thermostability.

As set forth above, polypeptides comprising a wild-type colicin-DNAse domain modified to comprise one or more mutations can comprise a wild-type CL7 DNAse domain (SEQ ID NO: 1), a wild-type CL2 DNAse domain (SEQ ID NO: 2), a wild-type CL8 DNAse domain (SEQ ID NO: 3) or a wild-type CL9 DNAse domain (SEQ ID NO: 4) with one or more mutations that reduces DNAse activity, that decreases DNA binding and/or increases thermostability of the polypeptide. For example, and not to be limiting, the polypeptide can comprise SEQ ID NO: 1 with one or more mutations, wherein the one or more mutations are at one or more amino acids selected from the group consisting of R2, K4, K11, K45, K51, K52, H99, S105, H124 and H128. Optionally, the one or more mutations comprise one or more mutations selected from the group consisting of R2S/Z, K4E/Z, K11E/Z, K45E/Z, K51E, K52T/Z, H99N/Z, S105E/Z, H124N/Z and H128E/Z mutation in SEQ ID NO: 1, wherein Z is any natural amino acid except for glycine (G), cysteine (C), proline (P), lysine (K) or arginine (R). Optionally, the mutations comprise R2S, K4E, K11E, K45E, K51E, K52T, H99N, S105E, H124N and H128E mutations in SEQ ID NO: 1.

In another example, the polypeptide can comprise SEQ ID NO: 2 with one or more mutations, wherein the one or more mutations are at one or more amino acids selected from the group consisting of R2, K4, K11, K45, K51, K52, H99, S105, H124 and H128. Optionally, one or more amino acids at positions, 2, 4, 11, 45, 51, 52, 99, 105, 124 and 128 can be replaced with any natural amino acid except for glycine (G), cysteine (C), proline (P), lysine (K) or arginine (R). Optionally, the one or more mutations comprise one or more mutations selected from the group consisting of R2S, K4E, K11E, K45E, K51E, K52T, H99N, S105E, H124N and H128E. Optionally, the mutations comprise a R2S, a K4E, a K11E, a K45E, a K51E, a K52T, a H99N, a S105E, a H124N and a H128E mutation in SEQ ID NO: 2.

In another example, the polypeptide can comprise SEQ ID NO: 3 with one or more mutations, wherein the one or more mutations are at one or more amino acids selected from the group consisting of R2, K4, K11, K45, R51, K52, H99, S105, H124 and H128. Optionally, one or more amino acids at positions, 2, 4, 11, 45, 51, 52, 99, 105, 124 and 128 can be replaced with any natural amino acid except for glycine (G), cysteine (C), proline (P), lysine (K) or arginine (R). Optionally, the one or more mutations comprise one or more mutations selected from the group consisting of R2S, K4E, K11E, K45E, R51E, K52T, H99N, S105E, H124N and H128E. Optionally, the mutations comprise R2S, K4E, K11E, K45E, R51E, K52T, H99N, S105E, H124N and H128E mutations in SEQ ID NO: 3.

In another example, the polypeptide can comprise SEQ ID NO: 4 with one or more mutations, wherein the one or more mutations are at one or more amino acids selected from the group consisting of R2, K4, K11, K45, K51, A52, H99, S105, H124 and H128. Optionally, one or more amino acids at positions, 2, 4, 11, 45, 51, 52, 99, 105, 124 and 128 can be replaced with any natural amino acid except for glycine (G), cysteine (C), proline (P), lysine (K) or arginine (R). Optionally, the one or more mutations comprise one or more mutations selected from the group consisting of R2S, a K4E, a K11E, a K45E, a K51E, a A52T, a H99N, a S105E, H124N and H128E. Optionally, the mutations comprise R2S, K4E, K11E, K45E, K51E, A52T, H99N, S105E, H124N and H128E mutations in SEQ ID NO: 4.

Polypeptides that are at least about 80%, 85%, 90%, 95% or 99% identical to SEQ ID NO: 1, SEQ ID NO: 2, SEQ ID NO: 3 or SEQ ID NO: 4 can also be modified as set forth herein. For example, a polypeptide comprising an amino acid sequence that is at least about 80%, 85%, 90%, 95%, or 99% identical to SEQ ID NO: 1, SEQ ID NO: 2, SEQ ID NO: 3 or SEQ ID NO: 4 comprising one or more mutations at one or more amino acids selected from the amino acids at positions 2, 4, 11, 45, 51, 52, 99, 105, 124 and 128 are provided herein. For example, a polypeptide comprising an amino acid sequence that is at least about 80%, 85%, 90%, 95%, or 99% identical to SEQ ID NO: 1 comprising one or more mutations at one or more amino acids selected from the group consisting of R2, K4, K11, K45, K51, K52, H99, S105, H124 and H128 is provided herein. A polypeptide comprising an amino acid sequence that is at least about 80%, 85%, 90%, 95%, or 99% identical to SEQ ID NO: 2 comprising one or more mutations at one or more amino acids selected from the group consisting of R2, K4, K11, K45, K51, K52, H99, S105, H124 and H128 is also provided herein. Further provided is a polypeptide comprising an amino acid sequence that is at least about 80%, 85%, 90%, 95%, or 99% identical to SEQ ID NO: 3 comprising one or more mutations at one or more amino acids selected from the group consisting of R2, K4, K11, K45, R51, K52, H99, S105, H124 and H128. Also provided is a polypeptide comprising an amino acid sequence that is at least about 80%, 85%, 90%, 95%, or 99% identical to SEQ ID NO: 4 comprising one or more mutations at one or more amino acids selected from the group consisting of R2, K4, K11, K45, K51, A52, H99, S105, H124 and H128.

Those of skill in the art readily understand how to determine the identity of two polypeptides or nucleic acids. For example, the identity can be calculated after aligning the two sequences so that the identity is at its highest level. Another way of calculating identity can be performed by published algorithms. Optimal alignment of sequences for comparison can be conducted using the algorithm of Smith and Waterman Adv. Appl. Math. 2:482 (1981), by the alignment algorithm of Needleman and Wunsch, J. Mol. Biol. 48:443 (1970), by the search for similarity method of Pearson and Lipman, Proc. Natl. Acad. Sci. U.S.A. 85:2444 (1988), by computerized implementations of these algorithms (GAP, BESTFIT, FASTA, and TFASTA in the Wisconsin Genetics Software Package, Genetics Computer Group, 575 Science Dr., Madison, WI; the BLAST algorithm of Tatusova and Madden FEMS Microbiol. Lett. 174:247-250 (1999) available from the National Center for Biotechnology Information (http://www.ncbi.nlm.nih.gov/blast/b12seq/b12.html), or by inspection.

The polypeptides comprising a wild-type colicin-DNAse domain modified to comprise one or more mutations, can further comprise a cleavable polypeptide sequence in operable linkage with the colicin-DNAse domain, wherein the cleavable polypeptide sequence is at least about fifty amino acids in length. For example, the cleavable polypeptide sequence can be about 10 to about 75 amino acids in length, or greater. For example, the cleavable polypeptide sequence can be about 10, 15, 20, 25, 30, 35, 40, 45, 50, 55, 60, 65, 70, 75 amino acids or greater. Optionally, the cleavable polypeptide sequence comprises SEQ ID NO: 5. Optionally the cleavable polypeptide sequence is resistant to cellular proteases. The cleavable polypeptide sequence can be operably linked to the N-terminus or the C-terminus of the modified colicin-DNAse domain and can be used to link the modified colicin-DNAse domain with other polypeptide sequences, including a protein of interest, for example, a heterologous protein.

Optionally, the cleavable polypeptide sequence comprises a protease cleavage site and a histidine tag sequence. Examples of protease cleavage sites include, but are not limited to, a PreScission protease cleavage site (LEVLFQGP) (SEQ ID NO: 22) that is cleaved by PreScission protease (GE Life Sciences, Pittsburgh, PA), a SUMO domain that is cleaved by SUMO protease or a TEV protease cleavage site (for example, EXXYXQG/S) (SEQ ID NO: 23), wherein X is any natural amino acid). The histidine tag sequence can comprise about four to about ten histidine residues in succession. For example, the histidine tag sequence can be four, five, six, seven, eight, nine or ten consecutive histidine residues.

Optionally, the polypeptides comprising a wild-type colicin-DNAse domain are modified to comprise one or more mutations and include a cleavable polypeptide sequence in operable linkage with the colicin-DNAse domain. For example, the polypeptide comprises SEQ ID NO: 6 (a modified CL7 DNAse domain in operable linkage with a cleavable polypeptide sequence), SEQ ID NO: 7 (a modified CL2 DNAse domain in operable linkage with a cleavable polypeptide sequence), SEQ ID NO: 8 (a modified CL8 DNAse domain in operable linkage with a cleavable polypeptide sequence) or SEQ ID NO: 9 (a modified CL9 DNAse domain in operable linkage with a cleavable polypeptide sequence).

Optionally, the polypeptides comprising a wild-type colicin-DNAse domain modified to comprise one or more mutations and a cleavable polypeptide sequence in operable linkage with the colicin-DNAse domain can further comprise a heterologous protein operably linked to the cleavable polypeptide sequence, wherein the cleavable polypeptide sequence links the heterologous polypeptide with the colicin-DNAse domain. In the polypeptides comprising a heterologous protein, the cleavable polypeptide sequence can link the modified colicin-DNAse domain to the N-terminus of the heterologous protein or to the C-terminus of the heterologous protein.

As used throughout, a heterologous protein is a protein that is not naturally associated with a wild-type colicin-DNAse domain or portion thereof. Generally, the heterologous protein is not normally produced by a cell in which the nucleic acid encoding a polypeptide comprising the heterologous protein is introduced. However, the heterologous protein can be naturally produced by a cell in which a nucleic acid encoding a polypeptide comprising the heterologous protein is introduced, such that the protein is both produced naturally and recombinantly by the cell.

The heterologous protein can be a eukaryotic or prokaryotic protein. The heterologous protein can be a full-length protein or a fragment thereof. The heterologous protein can be from a pathogen, such as, a parasite, a fungus, a bacteria, a virus or a prion. The heterologous protein can be a cytoplasmic protein, a membrane protein or a multi-subunit protein. The heterologous protein can be an enzyme, a hormone, a growth factor, a cytokine, an antibody or a portion thereof, a structural protein or a receptor, to name a few. The heterologous protein can also be a vaccine protein or a protein that is specifically expressed in a disease state, for example, a cancer-specific protein.

### Polypeptides Comprising a Modified Colicin Immunity Protein

Further provided herein are polypeptides comprising a colicin immunity protein, wherein the immunity protein comprises one or mutations that increase thermostability of the polypeptide. Optionally, the colicin immunity protein is Im7 with one or more mutations. For example, the polypeptide can comprise SEQ ID NO: 10 with one or more mutations. Optionally, the polypeptide comprises SEQ ID NO: 10 with one or more mutations at one or more amino acid positions selected from the group consisting of L3, K4, A13, Q17, K20, E21, K24, V33, V36, L37, K43, K70, K73, A77 and K81. Optionally, the one or more mutations are selected from the group consisting of L3F, K4R, A13E, Q17R, K20R, E21G, K24R, V33R, V36W, L37M, K43E, K70E, K73R, A77E and K81R in SEQ ID NO: 10. Optionally, the mutations comprise L3F, K4R, A13E, Q17R, K20R, E21G, K24R, V33R, V36W, L37M, K43E, K70E, K73R, A77E and K81R in SEQ ID NO: 10.

Optionally, the colicin immunity protein is Im9 with one or more mutations. For example, the polypeptide can comprise SEQ ID NO: 11 with one or more mutations. Optionally, the polypeptide comprises SEQ ID NO: 11 with one or more mutations at one or more amino acid positions selected from the group consisting of L3, K4, A5, A13, Q17, T21, K35, L36, M43, K57, Q72, A76, K80 and K84. Optionally, the one or more mutations are selected from the group consisting of L3F, K4R, A5D, A13E, Q17R, T21S, K35W, L36M, M43I, K57R, Q72R, A76E, K80R and K84Q in SEQ ID NO: 11. Optionally, the mutations comprise L3F, K4R, A5D, A13E, Q17R, T21S, K35W, L36M, M43I, K57R, Q72R, A76E, K80R and K84Q in SEQ ID NO: 11.

The polypeptides comprising a modified colicin immunity protein can further comprise a cleavable polypeptide sequence in operable linkage with the colicin immunity protein domain. The cleavable polypeptide sequence is descrbied above. The cleavable polypeptide sequence can be operably linked to the N-terminus or the C-terminus of the modified immunity protein. Optionally, the cleavable polypeptide sequence comprises a protease cleavage site and a histidine tag sequence. Examples of protease cleavage sites and histidine tag sequences are described above.

Optionally, the polypeptide further comprises a polypeptide sequence comprising a thioredoxin tag, wherein the cleavable polypeptide sequence links the thioredoxin tag and the colicin immunity protein. Optionally, the polypeptide further comprises amino acid sequences comprising a cysteine-containing coiled-coil, wherein the amino acid sequences flank the colicin immunity protein. By flanking is meant immediately adjacent on each end of the colicin immunity protein or juxtaposed in close proximity. Optionally, the polypeptide comprises SEQ ID NO: 12 or SEQ ID NO: 13.

The polypeptides comprising a modified colicin immunity protein and/or the polpeptides comprising a DNAse domain can be immobilized on a solid support. For example, and not to be limiting, the solid support can be a magnetic bead, an agarose-based resin or an agarose bead. In other examples, the solid support comprises non-agarose chromatography media, monoliths or nanoparticles. For example, the chromatography media can be, e.g., methacrylate, cellulose, or glass. In other examples, the nanoparticles are gold nanoparticles or magnetic nanoparticles.

Further provided is an affinity matrix comprising a substrate and one or more of the polypeptides provided herein, wherein the one or more polypeptides are conjugated or crosslinked to the substrate. For example, one or more polypeptides, or a plurality of polypeptides comprising a modified immunity protein, can be conjugated to the substrate. The substrate can, for example, a magnetic bead, an agarose-based resin or an agarose bead.

### Purification Systems

The polypeptides comprising a modified colicin DNAse domain and the polypeptides comprising a modified immunity protein form a high affinity complex with a binding affinity that approaches the binding affinities of a covalent bond (Kₘ10⁻¹⁴ -10⁻¹⁷). For example, a modified CL7 DNAse domain specifically binds to a modified Im7 protein. In another example, a modified CL9 DNAse domain specifically binds to an Im9 protein.

The purification methods provided herein rely on this interaction to efficiently purify heterologous proteins. When tagged heterologous proteins i.e., heterologous proteins linked by a cleavable polypeptide to polypeptides comprising a modified colicin DNAse domain, are contacted with polypeptides comprising a modified immunity protein, the modified colicin DNAse domain binds to the polypeptides comprising the modified immunity protein. Once bound to the polypeptides comprising the modified immunity protein, via the modified colicin DNAse domain, the heterologous protein can be cleaved from the complex by enzymatically cleaving the heterologous protein from the polypeptide comprising the modified colicin DNAse domain. Therefore, systems comprising a polypeptide comprising a modified colicin DNAse domain that specifically binds to polypeptides comprising a modified immunity protein are provided herein.

### Nucleic Acids

Further provided is a nucleic acid encoding any one of the polypeptides provided herein. Modifications in the amino acid sequences in the polypeptides provided herein can arise as allelic variations (e.g., due to genetic polymorphism), may arise due to environmental influence (e.g., due to exposure to ultraviolet radiation), or other human intervention (e.g., by mutagenesis of cloned DNA sequences), such as induced point, deletion, insertion, and substitution mutants. The mutations are not limited to the mutations described above, as additional modifications can be made. These modifications can result in changes in the amino acid sequence, provide silent mutations, modify a restriction site, or provide other specific mutations. Amino acid sequence modifications typically fall into one or more of three classes: substitutional, insertional, or deletional modifications. Insertions include amino and/or terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. Typically, no more than about from 2 to about 6 residues are deleted at any one site within the protein molecule. Amino acid substitutions are typically of single residues, but can occur at a number of different locations at once; insertions usually will be on the order of about from 1 to about 10 amino acid residues; and deletions will range from about 1 to about 30 residues. Deletions or insertions preferably are made in adjacent pairs, i.e., a deletion of 2 residues or insertion of 2 residues. Substitutions, deletions, insertions or any combination thereof can be combined to arrive at a final construct. The mutations may or may not place the sequence out of reading frame and may or may not create complementary regions that could produce secondary mRNA structure. Substitutional modifications are those in which at least one residue has been removed and a different residue inserted in its place.

Modifications, including the specific amino acid substitutions disclosed herein, are made by known methods. By way of example, modifications are made by site specific mutagenesis of nucleotides in the DNA encoding the polypeptide, thereby producing a DNA encoding the modification, and thereafter expressing the DNA in recombinant cell culture to produce the encoded polypeptides. Techniques for making substitution mutations at predetermined sites in DNA having a known sequence are well known, for example M13 primer mutagenesis and PCR mutagenesis.

The amino acids in the polypeptides described herein can be any of the 20 naturally occurring amino acids, D-stereoisomers of the naturally occurring amino acids, unnatural amino acids and chemically modified amino acids. Unnatural amino acids (that is, those that are not naturally found in proteins) are also known in the art, as set forth in, for example, Zhang et al. “Protein engineering with unnatural amino acids,” Curr. Opin. Struct. Biol. 23 (4): 581-587 (2013); Xie et la. “Adding amino acids to the genetic repertoire,” 9 (6): 548-54 (2005)); and all references cited therein. B and γ amino acids are known in the art and are also contemplated herein as unnatural amino acids.

As used herein, a chemically modified amino acid refers to an amino acid whose side chain has been chemically modified. For example, a side chain can be modified to comprise a signaling moiety, such as a fluorophore or a radiolabel. A side chain can also be modified to comprise a new functional group, such as a thiol, carboxylic acid, or amino group. Post-translationally modified amino acids are also included in the definition of chemically modified amino acids.

Also contemplated are conservative amino acid substitutions. By way of example, conservative amino acid substitutions can be made in one or more of the amino acid residues of any of the polypeptides provided herein. One of skill in the art would know that a conservative substitution is the replacement of one amino acid residue with another that is biologically and/or chemically similar. The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M)

By way of example, when an arginine to serine is mentioned, also contemplated is a conservative substitution for the serine (e.g., threonine). Nonconservative substitutions, for example, substituting a proline with glycine are also contemplated.

Those of skill in the art readily understand how to determine the identity of two polypeptides or nucleic acids. For example, the identity can be calculated after aligning the two sequences so that the identity is at its highest level. Another way of calculating identity can be performed by published algorithms. Optimal alignment of sequences for comparison can be conducted using the algorithm of Smith and Waterman, Adv. Appl. Math. 2:482 (1981); by the alignment algorithm of Needleman and Wunsch, J. Mol. Biol. 48:443 (1970); by the search for similarity method of Pearson and Lipman, Proc. Natl. Acad. Sci. U.S.A. 85:2444 (1988); by computerized implementations of these algorithms (GAP, BESTFIT, FASTA, and TFASTA in the Wisconsin Genetics Software Package, Genetics Computer Group, 575 Science Dr., Madison, WI; the BLAST algorithm of Tatusova and Madden FEMS Microbiol. Lett. 174:247-250 (1999) available from the National Center for Biotechnology Information (http://www.ncbi.nlm.nih.gov/blast/bl2seq/b12.html); or by inspection.

The same types of identity can be obtained for nucleic acids by, for example, the algorithms disclosed in Zuker, Science 244:48-52, 1989; Jaeger et al. Proc. Natl. Acad. Sci. USA 86:7706-7710, 1989; Jaeger et al. Methods Enzymol. 183:281-306, 1989 that are herein incorporated by this reference for at least material related to nucleic acid alignment. It is understood that any of the methods typically can be used and that, in certain instances, the results of these various methods may differ, but the skilled artisan understands if identity is found with at least one of these methods, the sequences would be said to have the stated identity.

For example, as used herein, a sequence recited as having a particular percent identity to another sequence refers to sequences that have the recited identity as calculated by any one or more of the calculation methods described above. For example, a first sequence has 80 percent identity, as defined herein, to a second sequence if the first sequence is calculated to have 80 percent identity to the second sequence using the Zuker calculation method even if the first sequence does not have 80 percent identity to the second sequence as calculated by any of the other calculation methods. As yet another example, a first sequence has 80 percent identity, as defined herein, to a second sequence if the first sequence is calculated to have 80 percent identity to the second sequence using each of calculation methods (although, in practice, the different calculation methods will often result in different calculated identity percentages).

### Vectors

Further provided is a vector comprising a nucleic acid set forth herein. The vector can direct the in vivo or in vitro synthesis of any of the polypeptides described herein. The vector is contemplated to have the necessary functional elements that direct and regulate transcription of the inserted nucleic acid. These functional elements include, but are not limited to, a promoter, regions upstream or downstream of the promoter, such as enhancers that may regulate the transcriptional activity of the promoter, an origin of replication, appropriate restriction sites to facilitate cloning of inserts adjacent to the promoter, antibiotic resistance genes or other markers which can serve to select for cells containing the vector or the vector containing the insert, RNA splice junctions, a transcription termination region, or any other region which may serve to facilitate the expression of the inserted gene or hybrid gene (See generally, Sambrook et al. Molecular Cloning: A Llaboratory Manual, 4^{th} ed., Cold Spring Harbor Laboratory Press, Cold Spring Harbor, 2012). The vector, for example, can be a plasmid. The vectors can contain genes conferring hygromycin resistance, ampicillin resistance, gentamicin resistance, neomycin resistance or other genes or phenotypes suitable for use as selectable markers, or methotrexate resistance for gene amplification.

There are numerous other E. coli expression vectors known to one of ordinary skill in the art, which are useful for the expression of the nucleic acid insert. Other microbial hosts suitable for use include bacilli, such as Bacillus subtilis, and other enterobacteriaceae, such as Salmonella, Senatia, and various Pseudomonas species. In these prokaryotic hosts, one can also make expression vectors, which will typically contain expression control sequences compatible with the host cell (e.g., an origin of replication). In addition, any number of a variety of well-known promoters will be present, such as the lactose promoter system, a tryptophan (Trp) promoter system, a beta-lactamase promoter system, or a promoter system from phage lambda. Additionally, yeast expression can be used. Provided herein is a nucleic acid encoding a polypeptide of the present invention, wherein the nucleic acid can be expressed by a yeast cell. More specifically, the nucleic acid can be expressed by Pichia pastoris or S. cerevisiae.

Mammalian cells also permit the expression of proteins in an environment that favors important post-translational modifications such as folding and cysteine pairing, addition of complex carbohydrate structures, and secretion of active protein. Vectors useful for the expression of active proteins in mammalian cells are known in the art and can contain genes conferring hygromycin resistance, genticin or G418 resistance, or other genes or phenotypes suitable for use as selectable markers, or methotrexate resistance for gene amplification. A number of suitable host cell lines capable of secreting intact human proteins have been developed in the art, and include the CHO cell lines, HeLa cells, COS-7 cells, myeloma cell lines, Jurkat cells, etc. Expression vectors for these cells can include expression control sequences, such as an origin of replication, a promoter, an enhancer, and necessary information processing sites, such as ribosome binding sites, RNA splice sites, polyadenylation sites, and transcriptional terminator sequences. Preferred expression control sequences are promoters derived from immunoglobulin genes, SV40, Adenovirus, Bovine Papilloma Virus, etc.

The expression vectors described herein can also include the nucleic acids as described herein and under the control of an inducible promoter such as the tetracycline inducible promoter or a glucocorticoid inducible promoter. The nucleic acids of the present invention can also be under the control of a tissue-specific promoter to promote expression of the nucleic acid in specific cells, tissues or organs. Any regulatable promoter, such as a metallothionein promoter, a heat-shock promoter, and other regulatable promoters, of which many examples are well known in the art are also contemplated. Furthermore, a Cre-loxP inducible system can also be used, as well as a Flp recombinase inducible promoter system, both of which are known in the art.

Insect cells also permit the expression of the polypeptides. Recombinant proteins produced in insect cells with baculovirus vectors undergo post-translational modifications similar to that of wild-type mammalian proteins.

### Cells

Also provided is a cell comprising a vector provided herein, wherein the cell is a suitable host cell for the expression of a nucleic acid encoding any of the polypeptides contemplated herein. The host cell can be a prokaryotic cell, including, for example, a bacterial cell. More particularly, the bacterial cell can be an E. coli cell. Alternatively, the cell can be a eukaryotic cell, including, for example, a Chinese hamster ovary (CHO) cell, a COS-7 cell, a HELA cell, an avian cell, a myeloma cell, a Pichia cell, an insect cell or a plant cell. A number of other suitable host cell lines have been developed and include myeloma cell lines, fibroblast cell lines, and a variety of tumor cell lines such as melanoma cell lines. The vectors containing the nucleic acid segments of interest can be transferred into the host cell by well-known methods, which vary depending on the type of cellular host. For example. calcium chloride transformation is commonly utilized for prokaryotic cells, whereas calcium phosphate, DEAE dextran, Lipofectamine, or lipofectin mediated transfection, electroporation or any method now known or identified in the future can be used for other eukaryotic cellular hosts.

### Compositions and Methods for Purification

Also provided herein is a method of purifying a heterologous protein comprising a) transfecting in a cell culture medium a cell with a vector, wherein the vector comprises a nucleic acid encoding a first polypeptide under conditions in which the first polypeptide is expressed, wherein the first polypeptide is a polypeptide comprising a heterologous protein and a wild-type colicin-DNAse domain modified to comprise one or more mutations, wherein the heterologous protein and the modified colicin-DNAse domain are linked by a cleavable polypeptide sequence; b) harvesting the cell culture medium comprising the expressed first polypeptide; c) lysing the cells to obtain a supernatant comprising the expressed first polypeptide; d) contacting the supernatant with an affinity matrix comprising a substrate and a second polypeptide, wherein the second polypeptide comprises a colicin immunity protein with one or more mutations, e) washing the matrix to remove biological molecules non-specifically bound to the first expressed polypeptide and the matrix; and f) eluting the heterologous protein from the matrix, comprising enzymatically cleaving the heterologous protein from the first polypeptide.

Also provided is a method of purifying a heterologous protein comprising a) transfecting in cell culture medium a cell with a vector comprising a nucleic acid encoding a first polypeptide under conditions in which the first polypeptide is expressed, wherein the first polypeptide is a polypeptide comprising a heterologous protein and a wild-type colicin-DNAse domain modified to comprise one or more mutations, wherein the heterologous protein and the modified colicin-DNAse domain are linked by a cleavable polypeptide sequence; b) harvesting the cell culture medium comprising the expressed first polypeptide comprising the heterologous protein; c) contacting the harvested cell culture medium with an affinity matrix, wherein the affinity matrix comprises a substrate and a second polypeptide comprising a colicin immunity protein with one or more mutations; d) washing the matrix to remove biological molecules non-specifically bound to the expressed first polypeptide and the matrix; e) eluting the heterologous protein from the matrix, comprising enzymatically cleaving the heterologous protein from the first polypeptide.

The cells that express the heterologous protein can be any cell that is suitable for the expression of the polypeptide comprising the heterologous protein, including any cell described herein. Heterologous proteins can also be purified from cells comprising a genome that has been genetically modified to express the polypeptide comprising the heterologous protein, i.e., a fusion protein comprising a modified colicin DNAse domain, a cleavable polypeptide linker and a heterologous protein.

Optionally, the purification methods can further comprise, after enzymatic cleavage of the heterologous polypeptide, eluting the first polypeptide, i.e., the modified colicin DNAse tag that is bound to the second polypeptide, i.e., the polypeptide comprising the modified colicin immunity protein, on the matrix and reactivating the matrix comprising the second polypeptide. For example, and not to be limiting, the first polypeptide can be eluted with 6M guanidine hydrochloride (G-HCl). The matrix comprising the second polypeptide, for example, an Im7 column can be reactivated by a one hour gradient refolding of Im7 during which where G-HCl is replaced with an appropriate buffer, as set forth in the Examples.

In the methods of purifying a heterologous protein, the cell culture medium comprising the expressed first polypeptide can be contacted with an affinity matrix comprising a second polypeptide comprising a colicin immunity protein under conditions that include salt concentrations of about 0.5M to about 2.0M. For example, the salt concentration can be about 0.5M, 0.6M, 0.7M, 0.8M, 0.9M, 1.0M, 1.1M, 1.2M, 1.3M, 1.4M, 1.5M or any concentration in between these concentrations. Salts such as NaCl and KCl can be used in any of the methods provided herein. Other salts are available to those of skill in the art. Based on the teachings of the specification, one of skill in the art would know how to select and use a salt and appropriate concentrations for purification of a protein of interest.

In the methods of purifying a heterologous protein, the affinity matrix can be any material to which a ligand, for example, a colicin immunity protein described herein, can be attached. The affinity matrix can include a solid support to which the colicin immunity protein can be attached. Examples of solid supports include, but are not limited to, beads, chips, capillaries or a filter comprising synthetic polymers (for example, polyvinyl alcohol, polyhydroxyalkyl acrylates, polyhydroxyalkyl methacrylates, polyacrylamides, polymethacrylamides), agarose, cellulose, dextran, polyacrylamide, latex or controlled pore glass.

In the methods of purifying a heterologous protein, the colicin immunity protein can be, for example, Im7 or Im9. Optionally, the polypeptide comprises SEQ ID NO: 10 with one or more mutations as described above.

Optionally, the colicin immunity protein is Im9 with one or more mutations as described above. For example, the polypeptide can comprise SEQ ID NO: 11 with one or more mutations as described above.

The polypeptides comprising a modified colicin immunity protein can further comprise a cleavable polypeptide sequence in operable linkage with the colicin immunity protein domain, wherein the cleavable polypeptide sequence is as described above. The cleavable polypeptide sequence can be operably linked to the N-terminus or the C-terminus of the modified immunity protein. Optionally, the cleavable polypeptide sequence comprises a protease cleavage site and a histidine tag sequence. Examples of protease cleavage sites and histidine tag sequences are described above.

Optionally, the polypeptide further comprises a polypeptide sequence comprising a thioredoxin tag, wherein the cleavable polypeptide sequence links the thioredoxin tag and the colicin immunity protein. Optionally, the polypeptide further comprises amino acid sequences comprising a cysteine-containing coiled-coil, wherein the amino acid sequences flank the colicin immunity protein. Optionally, the polypeptide as used in the methods comprises SEQ ID NO: 12 or SEQ ID NO: 13.

### Genetic Modification of Cells

Provided herein is a method of genetically modifying the genome of a cell to encode a chimeric polypeptide comprising a protein of interest and a modified colicin DNAse domain comprising: a) introducing into a population of cells (i) a guide RNA (gRNA) comprising a first nucleotide sequence that hybridizes to a target DNA in the genome of the cell, wherein the target DNA is the coding sequence or a nucleic acid sequence adjacent to the N-terminus or the C-terminus of the coding sequence for the protein of interest, and a second nucleotide sequence that interacts with a site-directed nuclease; (ii) a recombinant site-directed nuclease, wherein the site-directed nuclease comprises an RNA-binding portion that interacts with the second nucleotide sequence of the gRNA, wherein the site-directed nuclease specifically binds and cleaves the target DNA to create a double-stranded break before the N-terminus or after the C-terminus of the coding sequence for the protein; and (iii) a donor nucleic acid sequence comprising (i) a third nucleotide sequence that encodes the polypeptide of any of claims 16-19 and (ii) a fourth nucleotide sequence that hybridizes to a genomic sequence flanking the double stranded break in the target DNA, wherein (a) (i), (a) (ii) and (a) (iii) are introduced into the cells under conditions that allow homology-directed repair and integration of the third nucleotide sequence into the target DNA to form a genetically modified cell.

Methods for site-specific modification of a target DNA in a population of cells are known in the art. For example, the nuclease, guide RNA and donor nucleic acid sequence can be introduced into the cells under conditions that allow homology-directed repair (HDR) and integration of a donor nucleotide, for example, a ssODN or double stranded nucleotide sequence into the target DNA. The nuclease, guide RNA and donor nucleic acid sequence can be introduced into the cell via nucleoporation. Methods for nucleoporation are known in the art. See, for example, Maasho et al. “Efficient gene transfer into the human natural killer cell line, NKL, using the amaxa nucleofection system,” Journal of Immunological Methods 284 (1-2): 133-140 (2004); and Aluigi et al. “Nucleofection is an efficient non-viral transduction technique for human bone marrow derived mesenchymal stem cells,” Stem Cells 24 (2): 454-461 (2006)), both of which are incorporated herein in their entireties by this reference.

Optionally, in the methods of genetically modifying cells using a site-directed nuclease, (a) (i), (a) (ii), and (a) (iiii) are introduced into the population of cells by transfecting the cells with one or more vectors comprising the guide RNA, the nucleic acid sequence encoding the nuclease, and the donor nucleic acid. Optionally, the nuclease is Cas9. Methods for site-specific modification using CRISPR/Cas9 systems are known in the art (See, for example, Smith et al. “Efficient and allele-specific genome editing of disease loci in human iPSCs,” Mol. Ther. 23 (3): 570-7 (2015); and Jo et al. “CRISPR/Cas9 system as an innovative genetic engineering tool: Enhancements in sequence specificity and delivery methods,” Biochim Biophys Acta 1856 (2): 234-243 (2015)).

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed, that while specific reference of each various individual and collective combinations and permutations of these compounds may not be explicitly disclosed, each is specifically contemplated and described herein. For example, it is understood that when combinations, subsets, interactions, purification conditions etc. are disclosed in Examples I and II, that while specific reference of each various individual and collective combinations and permutations of these combinations, subsets, interactions, purification conditions may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXAMPLE I

Described herein are purification systems that employ a synthetic protein tag (a polypeptide comprising a modified colicin DNAse domain) that has extraordinary affinity for a single receptor protein (a colicin immunity protein). Two cross-resistant, affinity purification systems were developed using genetic modifications of ultra-high affinity homologous receptor/ligand (RC/LG) complexes. The affinity of these complexes (km˜10⁻¹⁴10⁻¹⁷ M) approaches that of a covalent bond, which makes this purification system a powerful and unique tool for purifying large proteins, in significant quantities, in a single step. With the systems provided herein, it is possible to isolate cellular components with a level of purity that allows mass spectroscopy analysis of the contents.

### Preparation of Colicin Immunity Protein 7 (Im7) Immobilization Unit (IU)

### Expression

E. coli, BL21 DE cells transformed with a vector comprising a nucleic acid encoding genetically modified Im7 were used. The cells were grown in about 1-2 liters of TB medium, at 37° C., to an OD of about 0.8-0.9. Then, the temperature was decreased to 18-20° C. and expression was induced with 0.1-1 mM IPTG and the cells were allowed to grow overnight (20-24 hrs). The culture was centrifuged for about 20 mins. at 6,000 g and the cell pellet was stored at −80° C.

### Purification

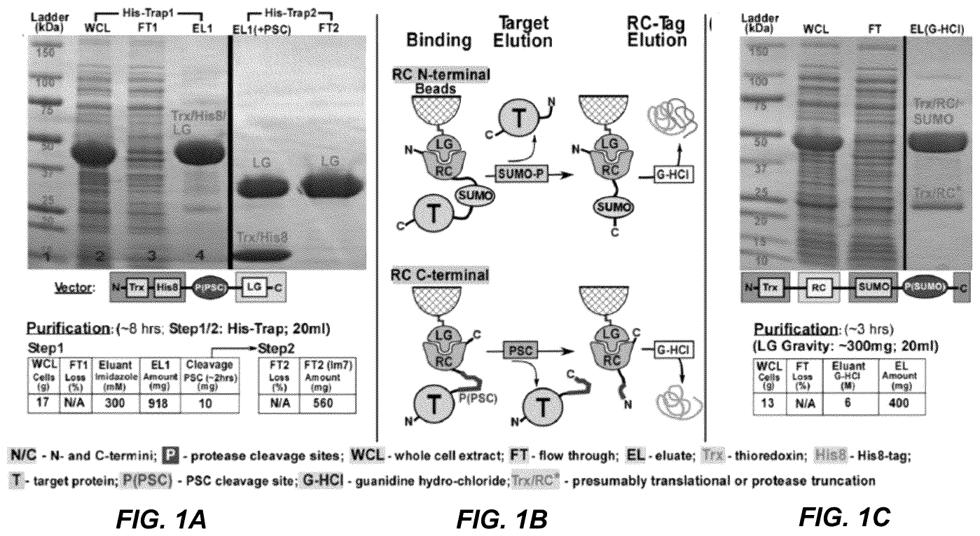

All procedures were carried out at 4° C. The cell pellet was suspended in lysis buffer (Buf-A; 0.5M NaCl 20 mM Tris pH 8, 5% Glycerol, 0.1 mM PMSF-added each 30 min) with a ratio of 10 ml Buf-A for 1 g cells and sonicated for about 30-40 mins, for about 100 ml of lysate. The lysate was heat sonicated at 70° C. for 45-50 min., and then centrifuged at 40,000 g for about 20 mins. Finally, the supernatant (SN) was filtered through a 45 μm filter. The filtered SN was loaded on the His-trap column (20 ml, GE Healthcare) at a flow rate of 5 ml/min with addition of 10-20 mM imidazole (IMZ). The column was washed with Buf-A in the presence of 30-50 mM IMZ and then the sample was eluted with 300 mM IMZ (FIG. 1A). The eluted sample was dialyzed against Buf-A with addition of His-tagged SUMO Protease (SUMO-P; ˜1 mg SUMO-P for 30-50 mg Im7 IU) for 4-6 hrs (or overnight). Then, the His-trap step was repeated, and the cleaved and ready to use Im7 IU was collected in a flow-through (FT) fraction (FIG. 1A).

### Immobilization of Im7 on Beads

SulfoLink coupling resin from Thermo Fisher was used according to the protocol provided by Thermo Fisher, as described below, (with scale up) (FIG. 1B) to covalently link the Im7 construct to agarose beads.

### Protocol for Immobilization (Thermo Fisher)

The peptide or protein to be immobilized must have free (reduced) sulfhydryls. Ellman's Reagent (Thermo Fisher, Product No. 22582) was used to determine if the peptide or protein contains free sulfhydryls. To make sulfhydryl groups available for coupling, disulfide bonds were cleaved with a reducing agent). If a sulfhydryl-containing reducing agent is used, desalting or dialysis is performed to remove the reducing agent before immobilization.

For peptide samples, Tris (2-carboxyethyl) phosphine (TCEP, Product No. 77720) efficiently reduces peptides but does not interfere with iodoacetyl coupling, requiring no removal of excess reagent before immobilization. TCEP is stable in aqueous solution and selectively reduces disulfide bonds. 0.1-1 mg of peptide are dissolved or diluted in 2 mL of Coupling Buffer and add TCEP to a final concentration of 25 mM TCEP.

For protein samples, 1-10 mg of protein are dissolved or diluted with 1mL of buffer (0.1M sodium phosphate, 5 mM EDTA-Na; pH 6.0). The protein solution is added to 6 mg of 2-MEA (50 mM). The mixture is incubated at 37° C. for 1.5 hours. 2-MEA is removed by performing two passes through a Thermo Scientific Zeba Spin Desalting Column (see Related Thermo Scientific Products) using the Coupling Buffer.

### Procedure for Immobilizing a Peptide or Protein Having Free Sulfhydryls

### Additional Materials Required

Column: Choose a glass or plastic column size appropriate for the volume of SulfoLink Resin to be used. The Disposable Column Trial Pack (Product No. 29925) contains accessories plus two each of three different column sizes, appropriate for 0.5-10 mL resin bed volumes. Alternatively, several centrifuge-ready Thermo Scientific Pierce Columns are available for resin bed volumes from 25 μl to 10 mL.
Coupling Buffer: 50 mM Tris, 5 mM EDTA-Na; pH 8.5. Prepare a volume equal to 20 times the volume of SulfoLink Resin to be used.
Quenching Reagent: L-cysteine.HCl (Product No. 44889)
Wash Solution: 1M sodium chloride (NaCl)
Storage Buffer: Phosphate-buffered saline (PBS) or other suitable buffer containing 0.05% sodium azide (NaN3)

### Preparation of SulfoLink Resin Column

SulfoLink Coupling Resin and all other reagents are equilibrated to room temperature. The bottle is stirred or swirled to evenly suspend the resin, and then a wide-bore pipette is used to transfer an appropriate volume of the 50% resin slurry to an empty column. For example, 2 mL of resin slurry is transferred to obtain a 1 mL resin bed. The column is then equilibrated with four resin-bed volumes of Coupling Buffer and the bottom column cap is replaced. When using gravity-flow columns, the resin bed does not become dry at any time throughout the procedure. More solution is added or the bottom cap on the column is replaced whenever the buffer drains down to the top of the resin bed.

### Couple Peptide/Protein to Resin

Prepared (i.e., reduced) peptide/protein is dissolved in Coupling Buffer and added to the column. 1-2 mL of peptide or protein solution per milliliter of SulfoLink Coupling Resin is used. If desired, a small amount of the peptide or protein solution is retained for later comparison to the coupling reaction flow-through fraction to estimate coupling efficiency. The top cap is replaced and the column is mixed (by rocking or end-over-end mixing) at room temperature for 15 minutes. The column is stood upright and incubated at room temperature for an additional 30 minutes without mixing. The top and bottom column caps are sequentially removed and the solution is allowed to drain from the column into a clean tube. The columns is placed over a new collection tube and the column is washed with three resin-bed volumes of Coupling Buffer. The coupling efficiency is determined by comparing the protein/peptide concentrations (e.g., by absorbance at 280 nm) of the noncoupled fraction to the starting sample.

### Block Nonspecific Binding Sites on Resin

The bottom cap on the column is replaced. A solution of 50 mM L-Cysteine.HCl is prepared in Coupling Buffer. One resin-bed volume of 50 mM cysteine solution is added to the column. This is mixed for 15 minutes at room temperature, and the reaction is incubated without mixing for an additional 30 minutes.

### Washing the Column

The top and bottom caps are sequentially removed and the column is allowed to drain. The column is washed with at least six resin-bed volumes of Wash Solution (1M NaCl) and then washed with two resin-bed volumes of degassed Storage Buffer.

### Preparation of Trx-CL7-SUMO Protein

### Expression

E. coli, BL21 DE cells were transformed with a vector comprising a nucleic acid encoding thioredoxin (Trx), a modified CL7 DNAse domain and a SUMO domain (cleaved by SUMO protease). The cells were grown in about 1-2 liters of TB medium, at 37° C., to an OD of about 0.8-0.9. Then, the temperature was decreased to 18-20° C. and expression was induced with 0.1-1 mM IPTG and the cells were allowed to grow overnight (20-24 hrs). The culture was centrifuged for about 20 mins. at 6,000 g and the cell pellet was stored at −80° C.

### Purification

All procedures were carried out at 4° C. The cell pellet was suspended in lysis buffer (Buf-A; 0.5M NaCl 20 mM Tris pH 8, 5% Glycerol, 0.1 mM PMSF-added each 30 min) with a ratio of 10 ml Buf-A for 1 g cells and sonicated for about 30-40 mins, for about 100ml of lysate. The lysate was heat sonicated at 70° C. for 45-50 min., and then centrifuged at 40,000 g for about 20 mins. Finally, the supernatant (SN) was filtered through a 45 μm filter. The filtered SN was loaded on the Im7 column (20 ml) at a flow rate of 2-4 ml/min. The column was washed with high salt (1.5M NaCl; 2-3 column volumes) followed by low salt (OM NaCl; 2-3 column volumes). The protein was eluted with 6M guanidine hydrochloride (G-HCl) (FIG. 1B). The Im7 column was reactivated with a one hour gradient refolding of Im7 where G-HCl was replaced with Buf-A (FIG. 1C). Reactivation restored 100% capacity as revealed by an identical purification yield of about 410 mg for the model proteins after the Im7 column was used over 100 times.

### Bacterial multi-subunit RNA polymerases (RNAPs)

Two RNAPs (5 protein subunits; α₂ββ′ω, MW ˜400 kDa) were expressed and purified via essentially the same expression and one step purification protocols—T. thermophilus (ttRNAP) and M. tuberculosis (mtRNAP). In these examples, the cleavable (by PreScission protease, PSC) CL7-tag is located at the C-terminal end of the largest β′-subunit of the RNAP. In both cases, the overall purification process takes about 4-5 hours, resulting in about 30 mg of catalytically active protein. By comparison, purification of untagged ttRNAP, of the same quality and yield, required five different chromatography columns and the process took about six to seven days.

### Expression

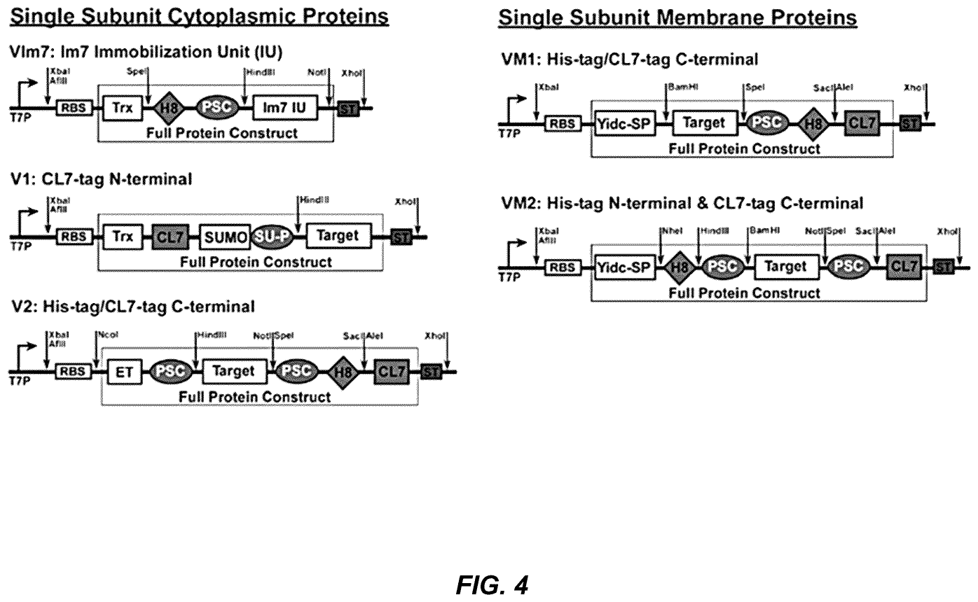

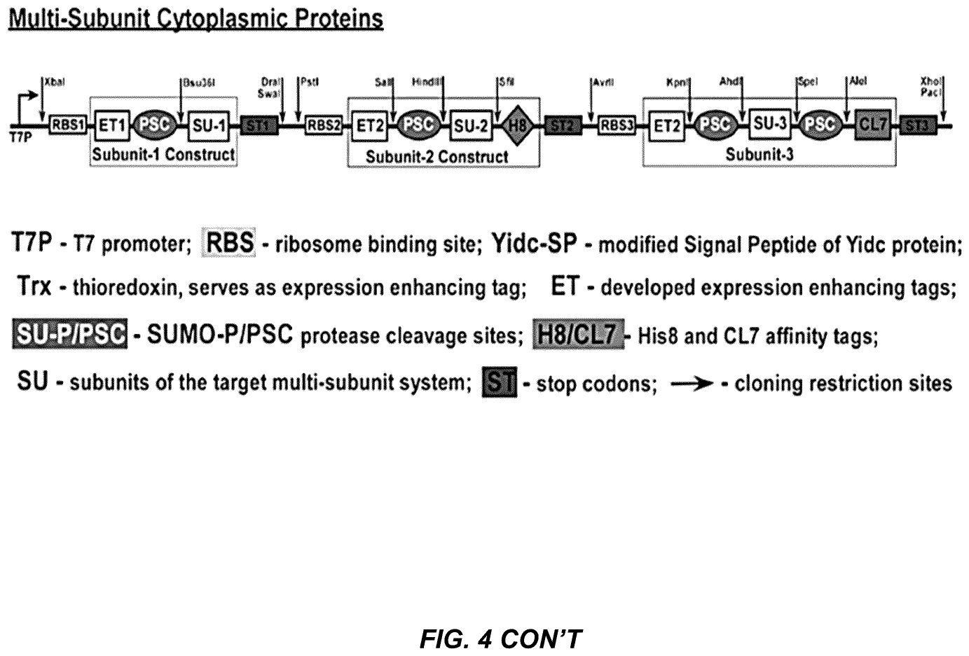

E. coli, BL21 DE or BL21 STAR cells transformed with a multisubunit vector comprising nucleic acids encoding subunits of ttRNAP or mtRNAP (See FIG. 4), were used. The cells were grown in about 1-2 liters of TB medium, at 37° C., to an OD of about 0.8-1.0.

Then, expression was induced with 0.1-1 mM IPTG and the cells were allowed to grow for 3-5 hours. The culture was centrifuged for about 20 mins. at 6,000 g and the cell pellet was stored at −80° C.

### Purification

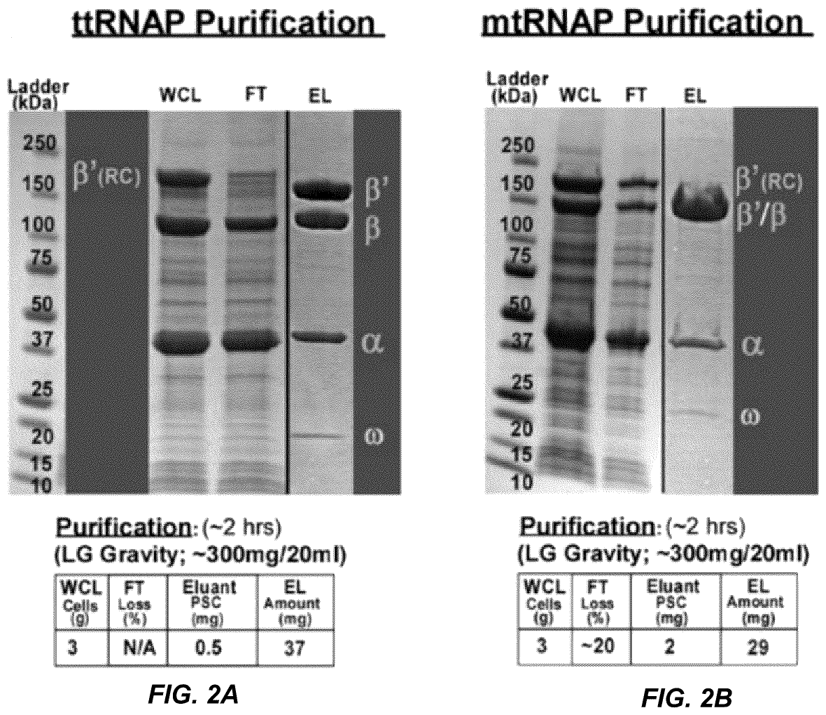

All procedures were carried out at 4° C. The cell pellet was suspended in lysis buffer (Buf-A; 0.5M NaCl 20 mM Tris pH 9, 5% Glycerol, 0.1 mM PMSF-added each 30′) with a ratio of 10 ml Buf-A for 1 g cells and the cells were disrupted with a French Press (about 16,000-20,000 psi, in 3 cycles). The lysate was centrifuged at 40,000 g for about 20 mins. Finally, the supernatant (SN) was filtered through a 45 μm filter. The filtered SN was loaded on the Im column (20 ml) at a flow rate of 2-4 ml/min. The column was washed with 2-3 alternate cycles of high salt (1.5M NaCl; 2-3 column volumes) followed by low salt (OM NaCl; 2-3 column volumes). Then, about 0.1-0.2 mg of PSC solution in Buf-A (for about 30-40 mg RNAP) was added. RNAP was eluted after about 1-3 hours (FIG. 2). The CL7-tag which remains bound to the Im7-column was washed out with 6M G-HCl. The Im7 column was reactivated with a 1 hour gradient replacing G-HCl for Buf-A.

### Transmembrane Proteins

Two trans-membrane proteins, bacterial membrane integrase Yidc (MW ˜32 kDa) and human chaperone Calnexin (CNX, ˜66 kDa) were expressed and purified via essentially the same expression and one step purification protocols. In these examples, the cleavable (by PSC) CL7-tag is located at the C-terminal end of each protein.

### Expression

E. coli, BL21 DE or BL21 STAR cells transformed with a single subunit membrane protein vector comprising a nucleic acid encoding nucleic acid encoding CNX or Yidc (See FIG. 4), were used. The cells were grown in about 1-2 liters of TB medium, at 37° C., to an OD of about 0.8-0.9. Then, the temperature was decreased to 18-20° C. and expression was induced with 0.1-1 mM IPTG. After induction, the cells were allowed to grow overnight (20-24 hours). The culture was centrifuged for about 20 mins. at 6,000 g and the cell pellet was stored at −80° C.

### Purification

All procedures were carried out at 4° C. The cell pellet was suspended in lysis buffer (Buf-A; 0.35M NaCl 20 mM Tris pH 8, 5% Glycerol, 0.1 mM PMSF-added each 30 min) with a ratio of 20 ml Buf-A for 1 g cells and the cells were disrupted with a French Press (about 16,000-20,000 psi, in 3 cycles). The lysate was centrifuged at 40,000 g for about 20 mins. Finally, the supernatant (SN) was filtered through a 45 μm filter.

DNA was eliminated using polyethylene-emine (PE) precipitation. Essentially, 0.06% PE was added to the lysate in 3 steps (0.02% PE for one step), mixing the precipitant for ˜5-10 min. between each step of PE addition. The PE-treated sample was centrifuged for 15-20 min at 4,000-8,000 g. The SN was disposed and the pellet was resuspended in Buf-A1 (0.6M NaCl, 20 mM Tris pH 8, 5% Glycerol, 1.5% dodecyl-maltopyranoside, DDM) and mixed for about 30-40 mins. The sample was centrifuged again and the salt concentration was increased to IM NaCl. The resulting SN was loaded on to the Im7 Column.

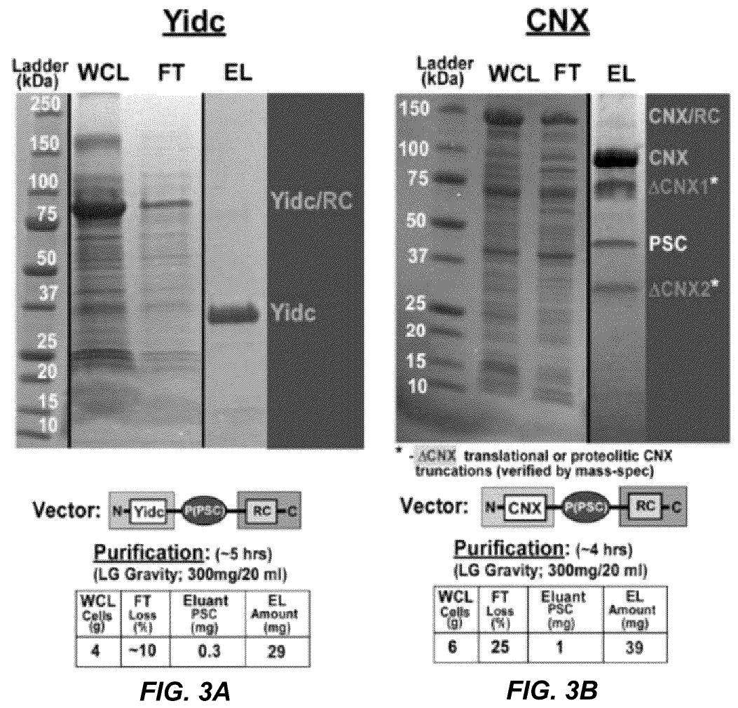

The PE-SN was loaded on the Im column (20 ml) at a flow rate of 1-2 ml/min. The column was washed with 2-3 alternate cycles of high salt (1.5M NaCl; 2-3 column volumes) followed by low salt (OM NaCl; 2-3 column volumes) buffers. Then, about 0.1-0.2 mg of PSC (for about 20-40 mg protein target) in Buf-A2 (0.5M NaCl, 20 mM Tris pH 8, 5% glycerol, 0.1% DDM) was added. The target protein was eluted after about 1-3 hours (FIG. 2). The CL7-tag which remains bound to the Im7-column was washed out with 6M G-HCl (FIG. 3). The Im7 column was reactivated with a 1 hour gradient replacing G-HCl for Buf-A.

### Purification of Condensin

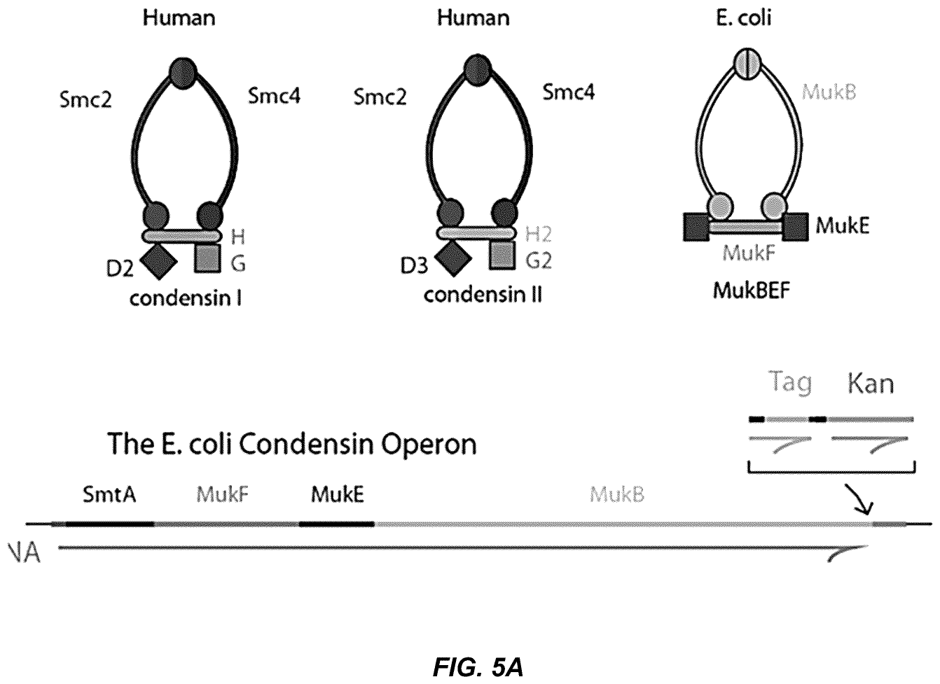

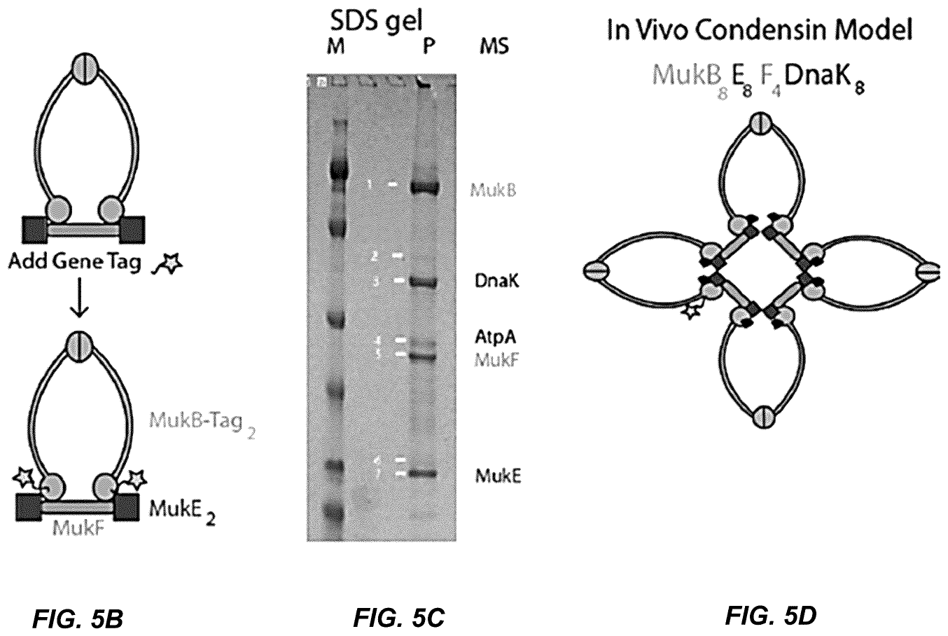

A Salmonella typhimurium DNA condensing complex, which was expected to have a MW of 583 kDa, turned out to have a molecular weight of >2,000 kDa. Condensins are highly conserved protein machines that fold and compact chromosomal DNA in bacterial and mammalian cells into self-adherent nucleoids and chromosomes, respectively. The structural similarity of bacterial and mammalian condensins is illustrated in FIG. 5A. In humans, there are two essential complexes. Condensin I and condensin II share a large subunit heterodimer of SMC2 and SMC4 but have different proteins associated with the terminal AAA-ATPase domain. In chromosome function condensin I compacts DNA along in the long axis while condensin II compacts DNA loops perpendicular to the long axis. In all organisms studied to date, condensins prove to be exceedingly difficult to reassemble from the known protein subunits. In E. coli and Salmonella the large subunit (MukB 175 kDa) is a homo-dimer that interacts with MukE and MukF via the AAA-ATPase motor. The mukB gene was modified in the chromosome by inserting the CL7 tag through a flexible protein linker to the C-terminus of the MukB (FIG. 5A). The expected structure of a MukFEB condensing is shown in FIG. 5B. 6 L of bacterial cells grown to late log phase (4 grams of packed cell paste) were prepared for cell lysis. Purification was carried out with a 1.5 ml mini-column of Im7 beads. After loading 200 ml of lysate onto the column, washing was carried out with 200 volumes of alternating high and low salt buffer. The column was eluted first by Prescission protease cleavage, which released only 20% of the protein, and then eluted with 6M G HCl, which released the >80%, which still retained at least one CL7 tag after proteolytic digestion. The isolated condensin complex was as pure as the Biorad markers for the condensin proteins (FIG. 5C). In addition to the expected proteins, MukB, MukE and MukF, two other proteins were detected. The molecular chaperone, DnaK was present in levels equivalent to MukB and MukE. This protein may be involved in changing a MukB dimer into a MukB octamer. A model for in vivo condensin structure of the 80% of the molecules retained on the column after proteolytic treatment is shown in FIG. 5D.

### Vectors

As shown in FIG. 4, numerous vectors have been constructed that allow expression and purification of single-subunit cytoplasmic and membrane proteins, as well as multi-subunit complexes. A pET28a vector from Novagen was used as the template for all of the vectors shown in FIG. 4. The nucleic acid sequence for the V1m7 vector is set forth herein as SEQ ID NO: 14. The nucleic acid sequence for the V1 vector is set forth herein as SEQ ID NO: 15. The nucleic acid sequence for the V2 vector is set forth herein as SEQ ID NO: 16. The nucleic acid sequence for the VM1 vector comprising a nucleic acid sequence encoding Yidc is set forth herein as SEQ ID NO: 17. The nucleic acid sequence for the VM2 vector comprising a nucleic acid sequence encoding CNX is set forth herein as SEQ ID NO: 18. The nucleic acid sequence for the V1m7 vector is set forth herein as SEQ ID NO: 19. The multi-subunit vector comprising a nucleic acid sequence encoding ttRNAP is set forth herein as SEQ ID NO: 20. The multi-subunit vector comprising a nucleic acid sequence encoding mtRNAP is set forth herein as SEQ ID NO: 21.

### EXAMPLE II

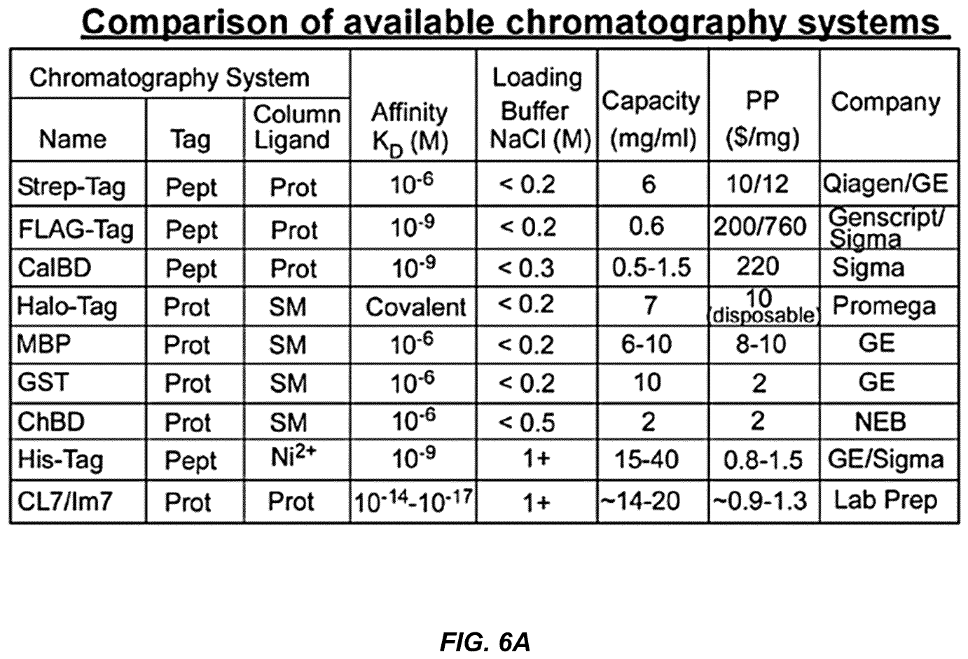

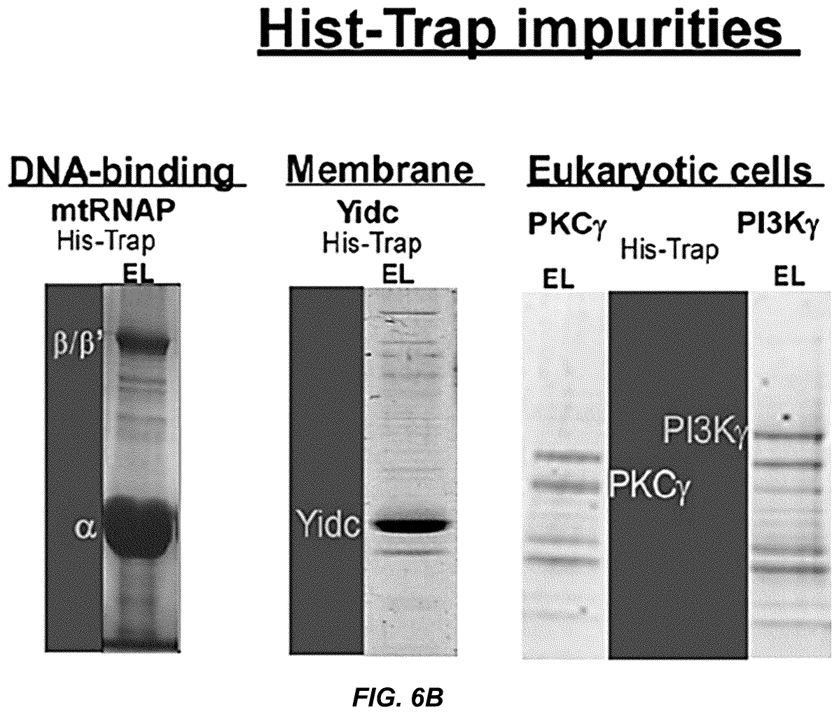

Biological approaches such as proteomics, interactomics and in vitro drug screening rely largely on efficient purification of the proteins being studied. Most widely used commercial columns provide modest yields of only a few milligrams (FIG. 6A). These yields are barely suitable for structural studies and commercial protein production. Moreover, analysis of the recommended commercial protocols and respective publications suggests that no commercial affinity system exists that tolerates high salt concentrations at the most crucial step of crude lysate loading on the columns (FIG. 6A). Most commercial systems tolerate a salt concentration of 0.2M-0.5M, at best. Consistently, trials with the GST, MBP and Step-Tag columns demonstrated that at salt concentrations over ˜0.3M only a very small fraction (5-10%) of the tagged proteins binds to the ligands on the column. On the other hand, loading the lysates at a low/medium salt concentration often results in residual contaminations that cannot be washed out entirely afterwards, even with high-salt buffers. Application of these “low-salt” columns, thus, is limited to only a subset of biological targets, which form no salt-dependent interactions with the pool of the untagged cellular molecules (for example, proteins, DNA/RNA).

In fact, among commercially available purification techniques only the His-tag (Ni²⁺-based) approach demonstrates not only H-yield capacity but also allows for high-salt loading of lysates without loss of binding affinities (FIG. 6A). However, results show that the His-tag approach possesses relatively high, non-specific, but cooperative affinity to DNA and to DNA-binding proteins, as well as to some cellular proteins, including many membrane proteins and cytosolic proteins from eukaryotic cells (FIG. 6B). These contaminations occur largely in a salt-independent manner that often result in only 30-70% purity for these, Ni²⁺ sensitive categories of biological targets (FIG. 6B), thus requiring additional purification steps to obtain high purity samples. Thus, protein purification remains a tricky, target-dependent, multi-step process that creates one of the major bottlenecks in biological sciences and their medical/industrial derivatives.

As set forth above, an ultra-high affinity purification system based on the small protein/protein (Colicin E7 DNAse/Immunity Protein 7; CL7/Im7; K_{D}˜10⁻¹⁴-10⁻¹⁷M) complex, with an affinity of 5-8 orders of magnitude higher than that of any other available analogs (FIG. 6A) allows for a one-step HHH-purification of the most challenging biological targets (for example, large multi-subunit, DNA/RNA-binding and membrane proteins).

### Expression

Unless otherwise specified the same procedures were used for plasmid construction, expression, cell growth and lysis throughout Example II. A commercial pET28 a expression vector (Invitrogen (Carlsbad, CA)) was used as a template vector and nucleotide gene sequences were inserted using the unique restriction sites of the vector. The gene sequences were designed through the manual inspection and modification of the natural (genomic) sequences to exclude the rare E. coli codons and high (G/C) content, where appropriate. The fragments of the designed sequences were synthesized commercially (IDT (Coralville, IA)) and then merged together, either through PCR (Phusion polymerase; NEB (Ipswich, MA) or through ligation using the unique restriction sites during cloning into the PET28a template vector. The resulting expression plasmids were transformed in BL21-Star (DE3) (Invitrogen) competent cells, colonies were grown overnight (37° C.) and several (2-3) resulting clones were sequenced to confirm that the sequences were correct. The cells were cultivated in TB media (http://www.bio-protech.com.tw/databank/DataSheet/Biochemical/DFU-J869.pdf) at 20° C. for 20-24 hrs in 2 or 4 liter flasks (for 1 or 2 L of culture) according to the following protocol.

The cells were first grown at 37° C. for ˜2-2.5 hrs until an OD₅₆₀ of the cultures reached values of ˜0.7-0.8. The temperature was then decreased to 20° C. and overexpression was induced by addition of 0.1 mM isopropyl β-D-1-thiogalactopyranoside (IPTG). The overnight cultures were centrifuged at 4,000 g for ˜30 minutes, and the cell pellets were frozen at −80° C. For purification, the frozen cell pellet was suspended in the respective lysate buffers (1 g cells→10 ml buffer) and then disrupted using the Nano DeBEE high pressure homogenizer (BEE International) using ˜15,000 psi of pressure for ˜3 mins (for ˜3 g cells) at 4° C. The lysates were then centrifuged at 40,000 g for 20 minutes and filtered through the 45 μm filter. All purifications were carried out using the Acta Prime purification system (GE Healthcare (Atlanta, GA)).

### Im7 Column Preparation

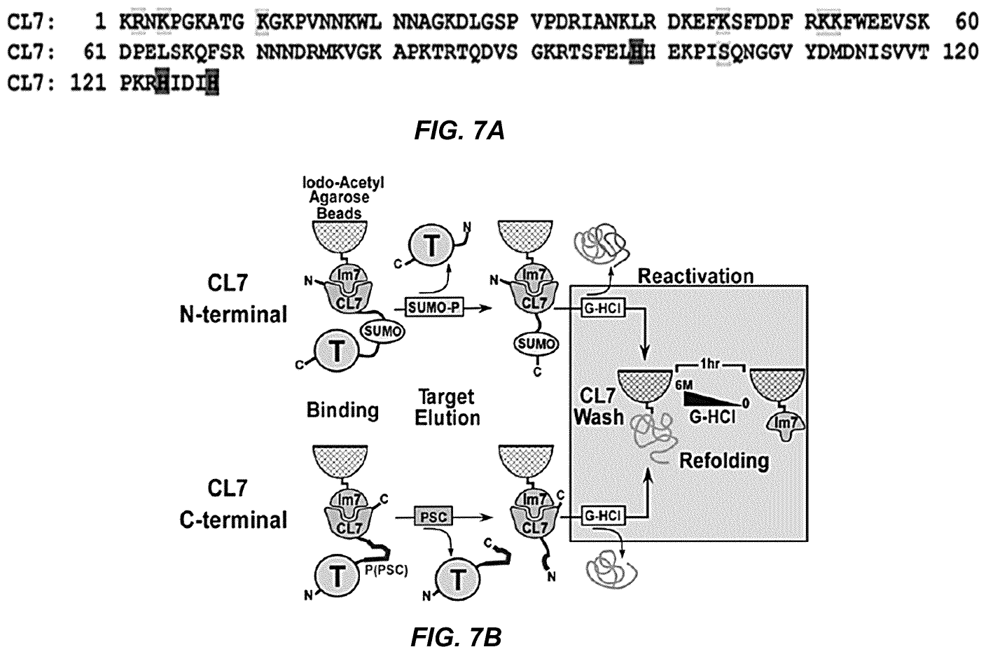

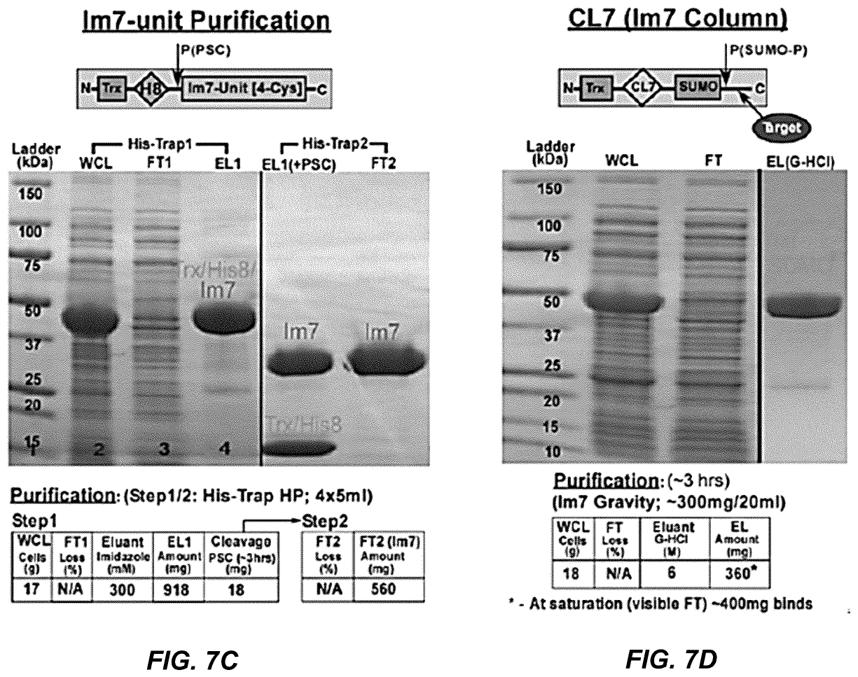

A 1 L culture of a Im7 immobilization unit usually produces ˜24 g of cells. Purification was carried out in two chromatographic steps (FIG. 7C). First, the cell lysate (lysis buffer: 0.5 M NaCl, 20 mM Tris pH 8, 5% glycerol, 0.1 mM PMSF) was loaded on the His-Prep FF (GE Healthcare) column (flow rate 5 ml/min.) in the lysis buffer (buffer A) with addition of 2% buffer B (1M imidazole). After loading, the column was washed by two alternate cycles of applying high/low (1M/OM NaCl) salt buffers with the addition of 5% buffer B, and then eluted in buffer A with addition of 25% buffer B. In the next step, the eluate was dialyzed against buffer A for ˜4-5 hrs in presence of purified prescission protease (PSC, lab prep) to cleave the expression (thioredoxin, Trx) and His8 tags (FIG. 7c). The dialyzed sample was heated at 70° C. for ˜45 min to eliminate PSC and was then loaded on the His-Prep column again under the same conditions as the first step. The flow through (FT) at the second step contained the very pure Im7-unit (FIG. 7C), which was then concentrated to ˜20-25 mg/ml and dialyzed against the coupling buffer recommended by Thermo Fisher (Waltham, MA) for protein immobilization to the Sulfo-Link (iodo-acetyl activated) 6B agarose beads. Immobilization was carried out according to the commercial protocol (http://www.funakoshi.co.jp/data/datasheet/PCC/20401.pdf) in a dark room and the reaction was normally completed in ˜20-25 min. The typical concentration of the immobilized Im7-unit was ˜15 mg/ml beads (or ˜0.8 mM). The Im7-activated beads were then packed in a 20 ml glass, low pressure column, which can be used for purification through respective adaptors, with the Acta Prime system.

### Purification of a Model Protein

To test column performance, a model protein (Trx→CL7→SUMO) was used that can also serve as a template for the target proteins with the N-terminal CL7-tag. In other words, a target protein can be cloned in this expression vector right after the SUMO domain using the unique HindIII/XhoI restriction sites. The Im7-column was tested with this model protein multiple times under different loading conditions varying salt (0.3-1.2M NaCl), reducing (β-mercaptoethanol up to 15 mM) and metal chelating (EDTA up to 20 mM) agents, detergent (DDM up to 1.5%) and flow rate (up to 4 ml/min.) and results similar to those shown in FIG. 7D were obtained. Upon loading, the column was subjected to a few (2-3) alternate cycles of high/low (1M/OM NaCl) salt buffer washing. The protein was then eluted under denaturing (6M guanidine hydrochloride, G-HCl) conditions, followed by column cleaning/reactivation using the gradient option of Acta Prime (FIG. 7B), i.e. gradually exchanging the G-HCl with the physiological buffer (0.5M NaCl, 20 mM Tris pH8, 5% glycerol) in ˜1 hr. No significant loss of capacity was detected after multiple (100+) reactivations. The concentrations of the model protein bound to the Im7 column were in the range of ˜(15-20) mg/ml beads.

### Purification of ttRNAP and mtRNAP

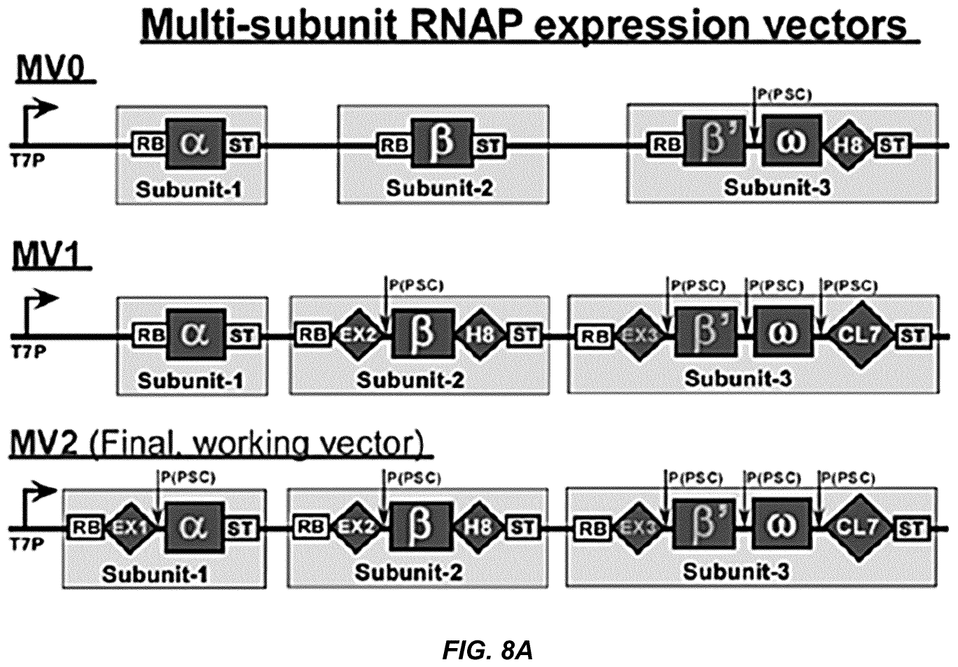

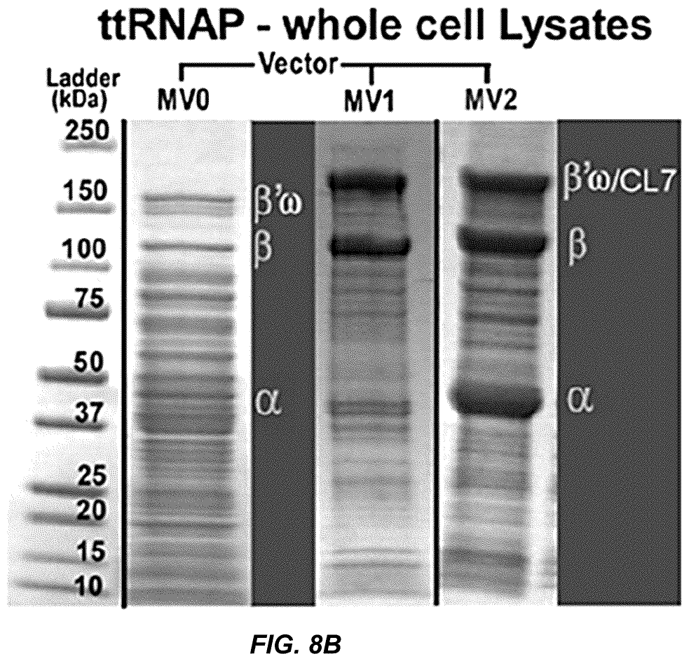

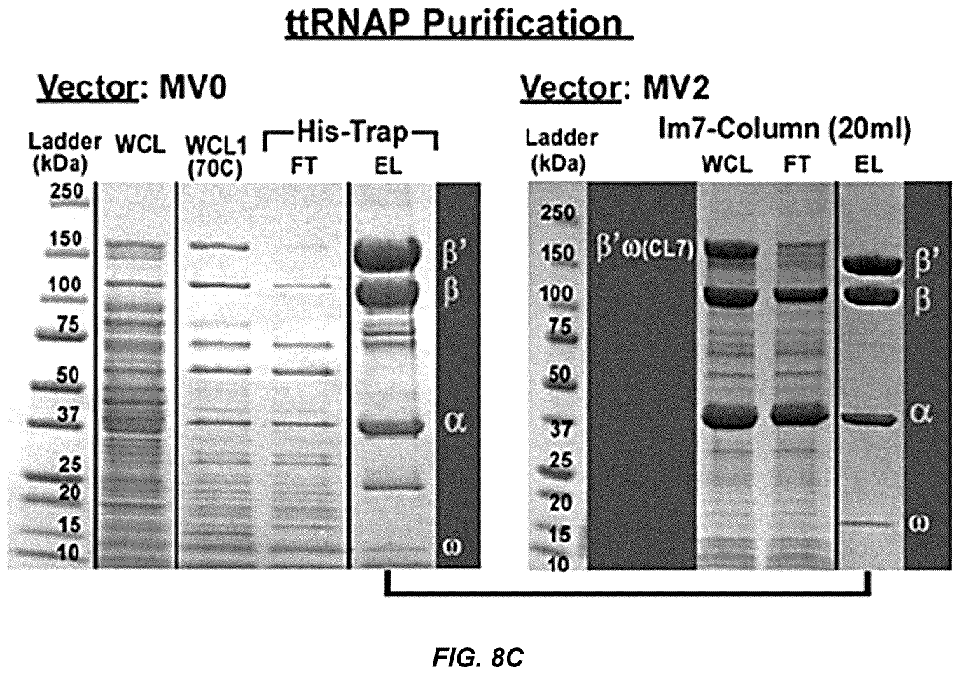

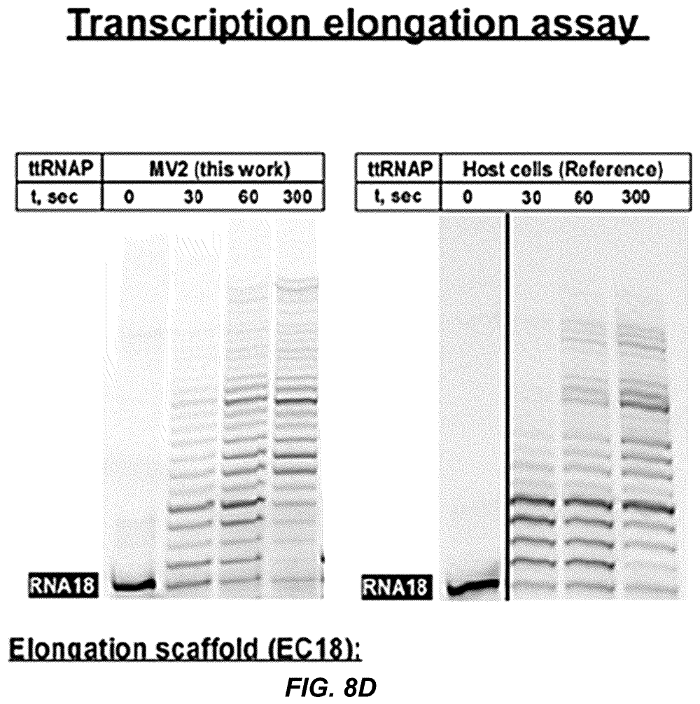

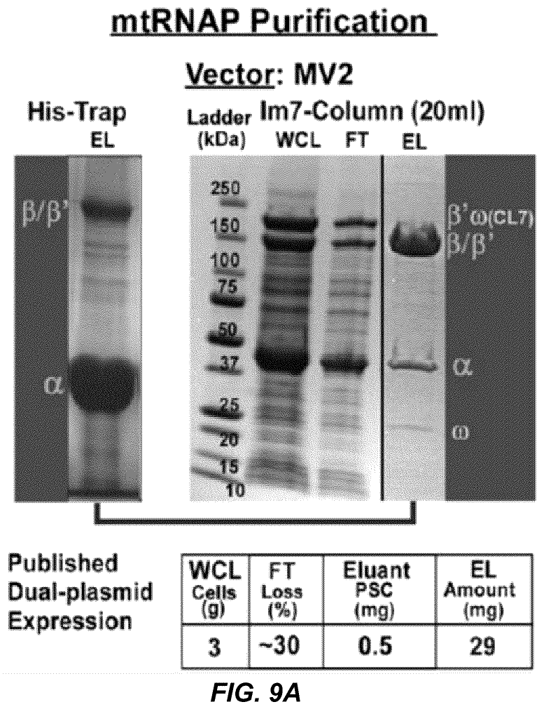

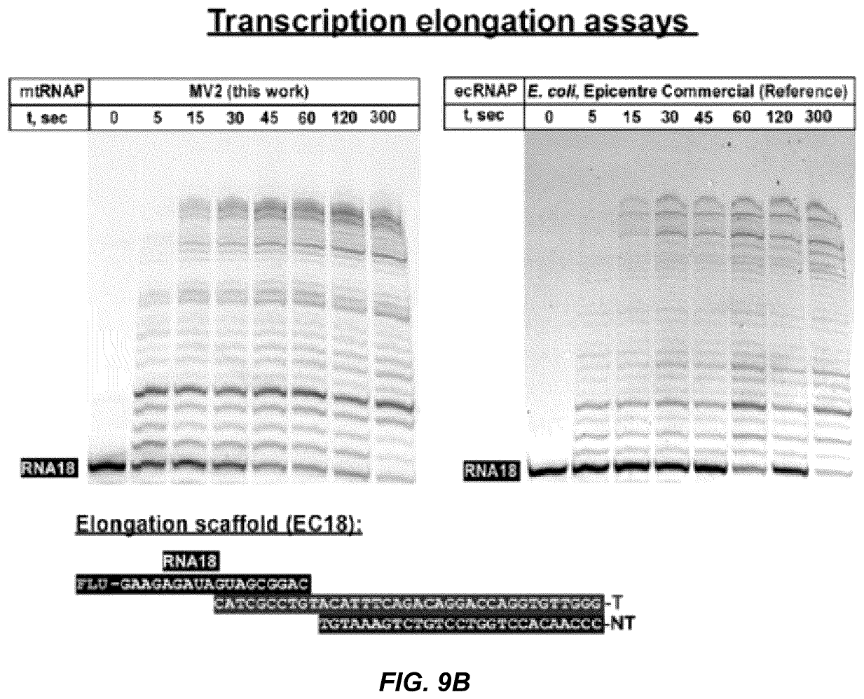

A 1 L culture of ttRNAP or mtRNAP usually produces ˜8-10 g cells. The lysis buffer contains 0.1M NaCl, 20 mM Tris pH 8.0, 5% glycerol, 0.5 mM CaCl₂, 10 mM MgCl₂, 0.1 mM PMSF, ˜120-150 μg DNAse I (Grade-I, Roche), and 1 tablet of an inhibitory cocktail for ˜3 g cells. The cell lysates were incubated for ˜1.5 hr at 4° C. in the lysis buffer, with addition of 0.05 mM PMSF each 30 mins. The lysates were then diluted 2 times with the 2-fold loading buffer containing (2.3M NaCl, 20 mM Tris pH 8.0, 5% glycerol) to increase salt concentration to 1.2M and loaded on the 20 ml Im7-column (flow rate of ˜1.5-2 ml/min; FIGS. 8C and 9A). Upon loading, the column was subjected to a few (2-3) alternate cycles of high/low (1M/0M NaCl) salt buffer washing. The proteins were then eluted using small amounts (˜0.3 mg) of PSC slowly (˜0.2 ml/min.) loaded on the column in the elution buffer (0.5M NaCl, 20 mM Tris pH 8.0, 5% glycerol, 0.2 mM EDTA). After elution the CL7-tag was washed out with 6M G-HCl and the column was reactivated through a standard gradient refolding procedure.

For the His-tagged ttRNAP construct (vector MV0, FIGS. 8A and 8C, left panel) purification was carried out under the same conditions as that for the Im7 purification, with the exceptions that 20 mM and 50 mM imidazole were added to the loading and washing buffers, respectively, while the protein was eluted with the elution buffer containing no EDTA and 250 mM imidazole.

### Fluorescent Transcription Assays

To assemble transcription elongation complexes, the 18-mer RNA (RNA18) labeled with fluoresceine (FLU) at the 5′-end, Template (T) and Non-Template (NT) oligonucleotides were ordered from IDT. The nucleic acid elongation scaffold was then assembled (FIGS. 8D and 9B). The transcription elongation reactions were carried out under the following conditions; 0.1M NaCl, 50 mM Tris pH 8.0, 5 mM MgCl2, 100 μM NTPs, ˜1.43 μM RNAP, 0.75 μM RNA18 (in the scaffold) at t=20° C. The reactions were stopped by 8M urea and loaded on the denaturing 20% polyacrylamide gels. The gels were then processed by the fluorescent imager, Typhoon Trio (GE Healthcare) and quantitated with the respective ImageQuant program (GE Healthcare).

### Purification of the Yidc Protein From the Uninduced Cells

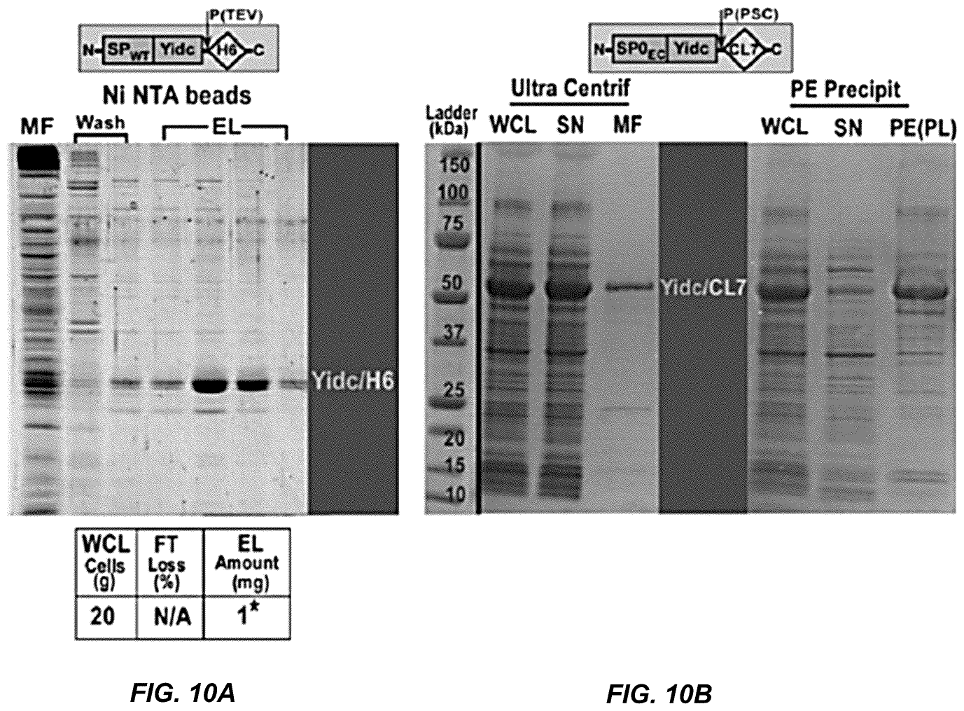

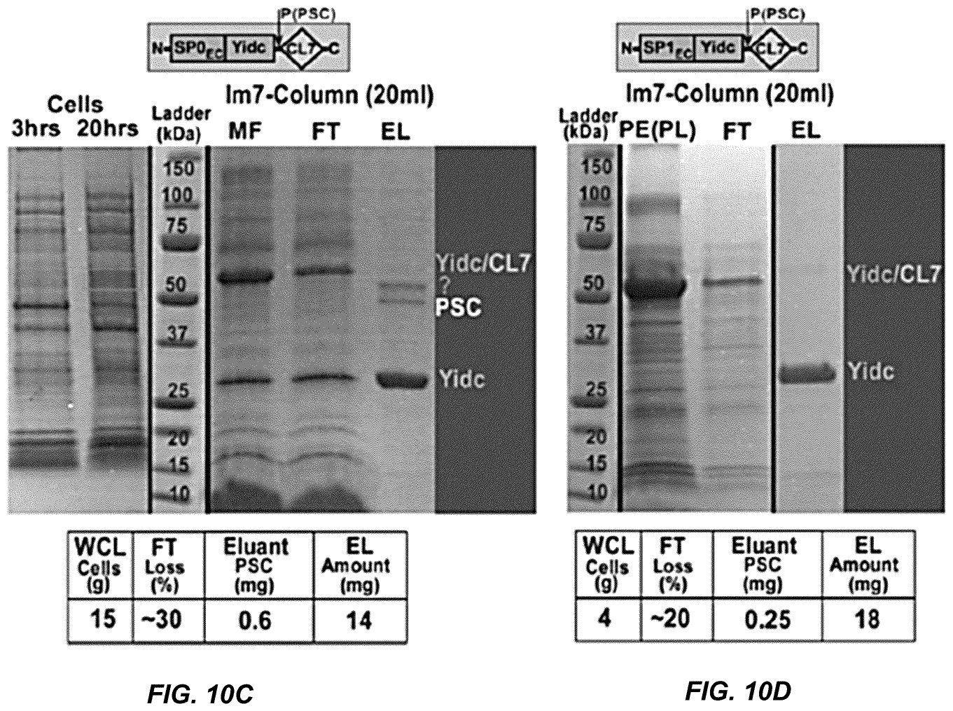

Cells were grown as described above, except that, after cell density reached OD₅₆₀˜ 0.7-0.8 at 37° C., the temperature was decreased to 20° C. with no IPTG addition. The 1 L culture of uninduced Yidc produced ˜20 g cells. The 200 ml of clear (filtered) lysate (lysis buffer: 0.5M NaCl, 20 mM Tris pH 8.0, 5% glycerol, 0.1 mM PMSF, 4 inhibitory tablets (Roche Catalogue No. 04 693 132 001 (Basel, Switzerland)) were ultracentrifuged at 120,000 g for 1.5 hrs. The pellet containing the membrane fraction (FIG. 10C) was then dissolved in ˜100 ml loading buffer (0.9M NaCl, 20 mM Tris pH 8.0, 5% glycerol, 0.1 mM PMSF, 1.5% DDM) and ultracentrifuged again at 120,000 g for 30 min. The supernatant was loaded on the 20 ml Im7-column (flow rate of ˜1.5 ml/min; FIG. 10C). Upon loading, the column was subjected to washing with a few (2-3) alternate cycles of high/low (1M/OM NaCl) salt buffers containing 0.1% DDM. The proteins were then eluted using the small amounts (˜0.6 mg) of PSC slowly (˜0.2 ml/min.) loaded on the column in the elution buffer (0.5M NaCl, 20 mM Tris pH 8.0, 5% glycerol, 0.2 mM EDTA, 0.1% DDM). After elution the CL7-tag was washed out with 6M G-HCl and the column was reactivated through the standard gradient refolding procedure (see above).

### Purification of the Yidc and Calnexin Proteins From the Induced Cells

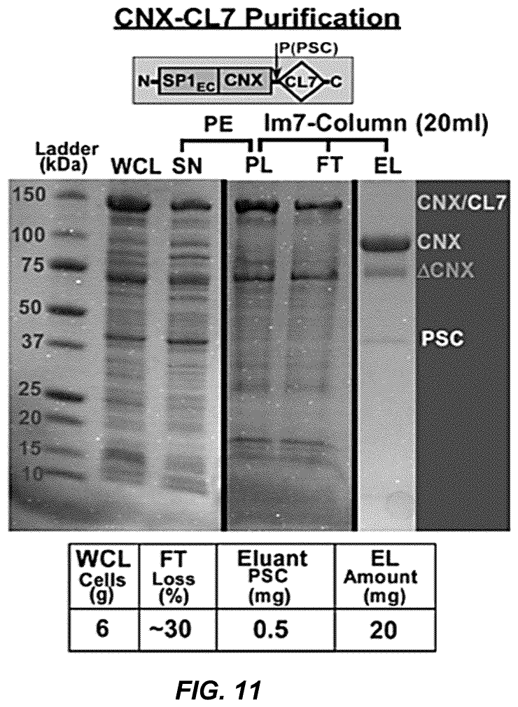

The cells for both the Yidc and calnexin (CNX) proteins were grown as described above with a standard over-expression induction (0.1 mM IPTG). The IL cultures of the induced Yidc and CNX produced ˜10 g cells. The lysates in the lysis buffers containing 0.35M/0.45M NaCl (for Yidc/CNX), 20 mM Tris pH 8.0, 5% glycerol, 0.1 mM PMSF, 1 inhibitory tablet) for ˜3 g cells were subjected to polyethylenimine (PE) precipitation (FIG. 10B, right panel, and (FIG. 11) according to the following protocol. The PE was added to lysates in the three steps (0.02% PE at each) to a final concentration of 0.06%. At each step, the lysates were gently mixed for ˜10mins. After the final step, the suspension was centrifuged at 5,000 g for 15 mins. A larger amount of the CNX protein (FIG. 11) remaining in the soluble fraction after the PE precipitation than that of Yidc (FIG. 10B) likely accounts for a higher salt concentration in the CNX lysate (0.45M vs 0.35M NaCl). The PE pellets were then washed with the solution containing 0.6M NaCl, 20 mM Tris pH 8.0, 5% glycerol and 1.5% DDM. The supernatant (soluble fraction) was diluted 10 times with the DDM-free high salt loading buffer to yield 0.9M NaCl, 20 mM Tris pH 8.0, 5% glycerol and 0.15% DDM. This diluted supernatant was loaded on the 20 ml Im7-column (flow rate of ˜1.5 ml/min; FIGS. 10D and 11). Upon loading, the column was subjected to washing with a few (2-3) alternate cycles of high/low (1M/0M NaCl) salt buffers containing 0.1% DDM. The proteins were then eluted using the small amounts (˜0.25 mg) of PSC slowly (˜0.2 ml/min.) loaded on the column in the elution buffer (0.5M NaCl, 20 mM Tris pH 8.0, 5% glycerol, 0.2 mM EDTA, 0.1% DDM). After elution the CL7-tag was washed out with 6M G-HCl and the column was reactivated through the standard gradient refolding procedure (see above).

### Results

### The (CL7/Im7) Chromatographic System

A family of colicins containing the DNAse domains (CL2, CL7, CL8, CL9) belong to a category of highly toxic enzymes. Their activity in host cells must be entirely suppressed, as this determines ultra-high affinity (4 to 7 orders of magnitude higher, for example, as compared to antibody/ligand complexes) to immunity proteins (Ims), the natural cognate inhibitors of the colicins. A number of other toxic enzymes are known and are characterized by similar ultra-high affinity to their inhibitors (for example, a group of colicins containing RNAse domains, eukaryotic DNAses, RNAses and proteases). All of them, therefore, can also be used as a basis for construction of ultra-high affinity columns. A major problem, however, is that the natural enzymes can be expressed only in the presence of their inhibitors. Their expression levels remain poor due to their toxicity, while their natural activities may affect purification of the target proteins. On the other hand, genetic inactivation of these enzymes is likely to result in substantial loss of affinity to their inhibitors, which in most cases target the enzymes' active sites. In this regard, the colicin DNAses (CLs) appear to be unique, since their cognate immunity proteins (Ims) bind remotely to the active/DNA-binding sites and sterically block DNA binding rather than activity of the enzymes. Using this unique feature of the (CL7/Im7) affinity pair and structural modeling, a CL7 variant, which entirely lacks catalytic and DNA binding activities (FIG. 7A) while retaining full binding affinity to its Im7 counterpart was constructed. CL7-tagged vectors were constructed. In these vectors, a target protein can be inserted either C-terminal (after the CL7 is fused to SUMO domain) or N-terminal to the CL7-tag, with the possibility for tag cleavage by the SUMO (SUMO-P) or prescission (PSC) proteases, respectively (FIG. 7B). An expression vector for large scale purification of the Im7-construct (FIG. 7C) extended with the non-functional coiled-coil domain containing 4 Cys-residues was also designed. This Im7-constuct was purified and covalently coupled to ˜20 ml of iodo-acetyl agarose beads (Sulfo-Link, Thermo Fisher). The expressed CL7-tagged proteins can be efficiently loaded on (bound to) a column from a lysate and washed under a variety of conditions. The target proteins are eluted from the CL7-column upon protease (SUMO-P/PSC) cleavage. The column is then cleaned/re-activated through elution of the CL7 tag under denaturing (for example, 6M guanidine-hydrochloride; G-HCl) conditions followed by ˜1 hr gradient refolding of Im7 (FIG. 7B). The Im7 column repeatedly allowed for large-scale purification (about 360-400 mg, nearly equimolar ratio) from the lysate of a model protein and possesses no non-specific binding (contaminations) to any of the untagged cellular components (FIG. 7D). The column restores ˜100% capacity upon re-activation, as revealed by a nearly identical purification yield for this model protein after the Im7 column was used 100+ times.

For the majority of the commercially available chromatographic systems, performance is typically determined based on purification of a limited number of the well-known, small or mid-size, stable and easily purified proteins. The actual performance, therefore, may drop dramatically once applied to the complex, non-trivial biological molecules. To avoid this technological caveat, for the studies described herein, complex, biologically significant targets were chosen from the three major categories. These targets are most refractory to HHH-purification for different reasons. A first group include large, multi-subunit proteins, which are often difficult to overexpress and purify due to potential truncations, large interacting area and flexible and/or poorly folded domains that increase the probability of non-specific interactions with cellular proteins, thus resulting in contaminations. A second group includes nucleic acid binding proteins that exhibit non-specific, yet significant (cooperative) DNA/RNA-binding affinities that cause major impurities and affect the binding capacity of a column. The membrane proteins constitute a third group of targets, for which the purification process is usually quite tricky and exhausting. Their hydrophobic nature results in non-specific binding to each other as well as cytosolic cellular proteins upon lysis, thus requiring the presence of high concentrations of detergents during purification to avoid contaminations. This group of proteins is also characterized by typically low overexpression levels that additionally complicate purification due to a poor signal-to-noise ratio in the crude cell extracts.

### Expression and Purification of DNA/RNA-Binding Bacterial Multi-Subunit RNA Polymerases

With respect to purification, multi-subunit RNA polymerases (RNAPs) are most complex since they combine characteristics of large multi-subunit and DNA/RNA-binding proteins. Two bacterial RNAPs core enzymes from evolutionary distinct organisms, T. thermophilus (ttRNAP) and pathogenic M. tuberculosis (mtRNAP) were selected for the following reasons. First, these proteins are very big (MW ˜400 kDa) multi-subunit protein complexes of five subunits (α₂ββ′ω). Stoichiometric expression is virtually impossible to achieve using the overexpression protocols in the E. coli host. The unbalanced expression creates the first line of complications for purification. Second, the largest β- and β′-subunits usually undergo transcription/translation-coupled truncations during overexpression. The resulting incomplete, loosely active RNAP molecules produce the second potential purification problem, which can only be resolved using affinity chromatography. Third, RNAPs contain at least four spatially distinct DNA-binding sites that are non-specific, but cooperatively bind strongly to cellular nucleic acids upon cell lysis. This results in major contamination by DNA/RNA and by DNA/RNA-binding proteins, respectively. In fact, after ˜2 hours of lysate (with overexpressed RNAPs) incubation in a medium (˜0.5M) salt buffer one can observe precipitation of the RNAP/DNA aggregates. These DNA/RNA-related impurities cannot be eliminated, for example, through a single His-Trap step, which is typically used for RNAP purification in the currently available overexpression systems, because His-Trap column itself possesses significant affinity to DNA (FIG. 6A and see below).

These impurities cannot be removed by any other single step chromatography, often requiring a time consuming, multi-step process that uses several columns, thus increasing time and effort. Also, this process often affects yield and activity of the final samples. Finally, multi-subunit RNAP is at the heart of transcription machinery, with high biological and medical significance. This protein has been extensively studied by various techniques, including high-resolution crystallographic analysis, for which HHH-purification is of central importance.

To establish an efficient protocol for the large-scale production and HHH-isolation of RNAPs, a multi-subunit, polycystronic expression vector following the two major criteria was designed. The vector can be used as a template for cloning of RNAPs from various species and presumably for other multi-subunit proteins, and enhance expression levels of the key (or each) individual subunits. A vector including ttRNAP was designed, since it was the most difficult target for overexpression in E. coli. Its nucleotide sequence is abundant of the E. coli rare codons and has exceedingly high G/C (˜70%) content, which together could result in the overall poor expression level coupled with many translational truncations. To minimize these potential challenges, the gene sequences of the RNAP subunits were manually designed and synthesized. Rare codons were eliminated, while the GC-content was decreased to a reasonable level for E. coli (about 59%). To test the expression performance of the vector as well as to have a reference point in purification for comparison with the developed (CL7/Im7) system, only a His-tag at the C-terminus of the largest β′-subunit was introduced. The resulting vector (FIG. 8A; MV0) demonstrated clear, yet modest expression levels of the intact RNAP molecules (FIG. 8B). However, multiple attempts of His-trap purification under the different loading/washing conditions failed to provide a reasonably pure sample even though a preliminary heated sample (free from most of the E. coli proteins) was used (FIG. 8C).

The vector was then modified to relocate the His-tag to the second largest β-subunit and to replace it with the CL7-tag (cleavable by PSC protease) at the C-terminus of the β′-subunit. In addition, the short N-terminal (also cleavable by PSC protease) “expression” tags were introduced. These tags were designed to increase the expression levels of each of the co-expressed subunits (FIG. 8A; MV2). For this, slightly modified (to avoid hydrophobic residues) short N-terminal sequences of the three proteins, known to improve expression when fused to the N-termini of the target proteins (FIG. 8A; MV2), were used. Assuming that these short peptides improve expression levels through stabilization of translation initiation as well as the respective full length proteins, a major advantage of this approach is that all tags (“expression” and purification) might be removed in a single step, by the same protease. The N-terminal peptide impurities can then be eliminated through a simple dialysis step from a final sample. The expression of all RNAP subunits was substantially improved, with the β′-subunit showing the lowest expression level of (FIG. 8B), which allowed use of the Im7-column as a first and only purification step to obtain the large amount (˜37 mg) of pure and intact RNAP sample (FIG. 8C). The activity tests using the synthetic elongation scaffold showed that the recombinant ttRNAP was as active as the one purified from the host thermophilic cells with ˜95-97% of the active (elongating) complexes (FIG. 8D).

After establishing efficient expression and one-step HHH-purification protocols for ttRNAP, the identical expression (FIG. 8A; MV2) and purification approaches were used to obtain essentially the same results, i.e. the enhanced expression levels and the HHH-sample obtained in one-step purification through the Im7 column (FIG. 9A) for mtRNAP. The purified enzyme possessed high (˜95%) activity in the elongation assay similar to, or better than that of commercial E. coli RNAP (FIG. 9B).

Notably, multiple purification trials with ttRNAP demonstrated that the best purity of ttRNAP, using both His-Trap and (CL7/Im7) approaches can be achieved if the lysates are first processed with DNAse and then loaded on the columns in high (1-1.2M) salt buffers. In particular, loading the lysates at a lower (0.5-0.8M) salt concentrations provided somewhat contaminated samples, even if the column was washed by the extra-high salt (2M) buffers after loading. Overall, these results showed that the HHH-samples of the big, multi-subunit DNA/RNA-binding proteins (RNAPs) can be obtained in one step and within only 5-6 hours. This demonstrates a dramatic improvement over the previously utilized approaches. For comparison, purification of untagged T. thermophilus RNAP from the host cells takes ˜8-10 days, requires up to 5 different columns, with a final yield of only ˜20 mg protein from ˜60 g cells. Large scale purification of His-tagged RNAPs also requires several (2-3) days and a number of distinct purification steps, which affect the final yield and often results in loss of activity of the enzyme.

### Expression and Purification of Membrane Proteins

Transmembrane proteins constitute up to 40% of a total protein pool in living cells. Most are of functional and clinical significance, yet only a few membrane proteins are well studied, much less studied at high-resolution. To a large extent, this deficit is related to challenges which occur at in the first, expression and/or purification steps of in vitro studies. The HHH-purification of membrane proteins required for crystallographic analysis is not trivial, mostly due to the unique hydrophobic nature and poor expression levels of the protein, even if an overexpression protocol is used. For these studies, two membrane proteins from different, prokaryotic and eukaryotic organisms, which also drastically differ in their configuration, size and function, were selected.

Bacterial Yidc membrane integrase (MW ˜32 kDa) is an all-membrane protein that contains no bulky outer-membrane domains. Its structure has been determined. This protein served as a reference in purification trials, as all parameters of traditional (His-Trap) purification were already known. According to the published results, the Yidc purification required three chromatographic steps (His-Trap1→[TEV-protease cleavage of His-tag]→His-Trap2→Gel-Filtration) to yield ˜1 mg pure protein from ˜15-20 g cells in ˜2-4 days. In particular, the first His-Trap step resulted in only ˜60% purity protein as the membrane proteins are known to have substantial binding affinities to the Ni²⁺-activated base (FIG. 10A). A poor final yield likely accounts not only for low overexpression levels that are typical for membrane proteins, but also for a loss of material during sequential purification steps.

In the studies described herein, a sequence of the Yidc gene was designed. This sequence was adjusted to E. coli codons and a vector was constructed with a PSC-cleavable CL7-tag fused at the C-terminus, as was done for the RNAPs (FIG. 10B). Surprisingly, this approach greatly improved the Yidc expression level as compared to published studies (FIG. 10A). Another surprise was that ultra-centrifugation, a traditional first step of the membrane proteins purification, which is always used to isolate the membrane fraction from the cytosolic proteins and nucleic acids, did not work at all. Not only was almost all of the overexpressed Yidc retained in a soluble fraction, but the pellet itself was very small and contained only a small amount of the cellular membrane proteins (FIG. 10B; left panel). The only explanation for this effect was that abundant overexpression of Yidc resulted in membrane fractionation upon lysis in the very small pieces, and the size/weight of the pieces did not allow them to precipitate, even under a huge, ultracentrifugal force. To check this possibility and to perform purification of Yidc under the standard, commonly used conditions, Yidc was expressed with no induction by IPTG since the vector that was used is characterized by promoter leaking. This approach solved the ultracentrifugation problem and allowed a high-purity Yidc sample to be obtained in one step using the Im7-column from the membrane fraction loaded on the column under high (0.9M) salt and detergent (dodecyl-maltopyranoside; DDM; 1.5%) concentrations. However, the amount of the purified sample was relatively modest (˜14 mg protein from ˜20 g cells), albeit much better than in the previously published work. Further, a small (˜5-7%) but visible impurity was observed on a final gel (FIG. 10C). This impurity might have accounted for the covalent linking of the protein to the membrane components through the Cys residue, which becomes the N-terminal residue upon cleavage by a signal peptidase. In previous studies, others introduced the TEV protease cleavage site immediately after this Cys residue to remove it after the first step of purification. In the studies described herein, the Cys residue was mutated to Ser, which often occurs in this (cleavage) position in signal peptides and, thus, should not affect the activity of a signal peptidase.

Studies were conducted to improve the purification yield using the lysate of the induced cells in purification, for which ultracentrifugation step was skipped. First, the cell lysate was loaded directly on the Im7-column, in essentially the same conditions that were used for membrane fraction purification. This run, however, resulted in a contaminated sample, in which, a substantial trace of DNA was observed, suggesting that the protein might have significant affinity to nucleic acids. Following this hypothesis, its DNA-binding affinity was analyzed through polyethylenimine (PE) precipitation and fit was found that Yidc precipitates almost entirely with DNA in ˜0.3-0.35 M salt (FIG. 10B; right panel). The following wash of the PE pellet in the higher-salt (0.6M) buffer containing 1.5% DDM fully released the Yidc protein in solution, while largely eliminating the nucleic acid component (FIG. 10B; right panel). Loading of the resulting supernatant on the Im7 column in high (˜0.9M) salt provided a high purity sample, but in relatively low quantity (˜4-5 mg from the 4 g cells). Most of it remained in the flow-through fraction. One possible reason for this might be in the high protein/DDM concentrations that cause a protein with micelles to form huge dynamic aggregates, both in solution and on a column to sterically block the Im7 active groups from the CL7-tag binding. To check this hypothesis, the lysate was diluted 10-fold with the detergent-free high (0.9M) salt buffer that greatly decreased both the protein and DDM concentrations. Though this approach increased purification time, it allowed for ˜4-fold improvement in yield (˜18 mg) from the same (4 g) amount of cells (FIG. 10D). The purified sample was also free of impurity observed in the protein with the wild-type signal peptide. At the same time, the size of the proteins (on a gel) were identical suggesting that the (C/S) mutation did not affect the signal peptide cleavage. Overall, these results provide a major improvement of Yidc purification over the previous, standard protocol (Kumakazi et al. “Crystallization and preliminary X-ray diffraction analysis of YidC, a membrane-protein chaperone and insertase from Bacillus halodurans,” Acta Crystallogr F Struct Biol Commun 70:1056-1060 (2014)).

A human calnexin (CNX) protein is a chaperone of substantially larger size (MW ˜65 kDa) than Yidc, which contains a short (˜35 residues long) transmembrane segment, and two soluble, outer membrane domains on both sides of the membrane. Markedly, full size CNX has never been overexpressed and purified in large quantities CNX likely forms a physiological complex with the HIV Nef protein in vivo. Therefore, this could be a promising target for anti-AIDS drug design. The detailed functional and structural studies of these interactions can be performed only in an in vitro model, for which HHH-purification of CNX is of central importance. To design the CNX overexpression vector, an identical purification approach was used. In particular, the natural CNX signal peptide was replaced with the mutated Yidc one, which could have accounted for improved expression levels of Yidc. Accordingly, the CNX expression level was high. The purification procedure, including the PE precipitation step, appeared to be very similar to that of Yidc and yielded ˜20 mg of high purity protein (FIG. 11). The only minor contamination (˜5-7%) observed in the purified CNX sample is not related to purification itself, but rather represents a truncated fragment of CNX. The binding activity of CNX to the Nef protein was further confirmed by the results of the surface plasmon resonance (SPR) and isothermal titration calorimetry (ITC) experiments. Notably, similar to that of RNAPs, loading of these membrane proteins on the Im7 column at high salt (about 0.9M) concentrations resulted in higher purity samples, since the lower salt concentrations produced contaminated proteins.

The protein purification systems provided herein allow high efficiency purification of proteins and large protein complexes from whole cell extracts using a single reusable chromatography column. The system employs a tag comprising a modified colicin DNAse domain that has extraordinary affinity for a colicin immunity protein. Genetic modifications eliminate the activities of the tag (colicin DNAse domain) except for its ability to bind, with high affinity to its receptor protein (colicin immunity protein). With the systems provided herein, it is possible to isolate cellular components with a level of purity that allows mass spectroscopy analysis of the contents. The immunity protein has been modified for efficient, covalent linkage to a broad range of solid supports, including, but not limited to, agarose beads and magnetic beads. Further, the tag (˜20 kDa) can be introduced at a C- or N-terminus of a wide range of proteins including membrane proteins. The one column system also works with high yield expressing plasmids to yield significant levels of multiprotein complexes (MPCs), which, until the present invention, have been difficult to purify in significant quantities. In addition, cells can be genetically modified, for example, using the CRISPR system, to genetically fuse a nucleic acid encoding the tag directly to a nucleic acid encoding a target protein in eukaryotic cells. The systems provided herein can be used to isolate a variety of proteins and MPCs from a single tissue, tissue culture preparations or genetically modified cells._ These studies also show that the ultra-high affinity (CL7/Im7) approach can facilitate studies of protein-protein interactions by surface plasmon resonance (SPR) spectroscopy, an essential technique for characterizing binding partners in physiological complexes and to validate results of foreseeable drug screening. The SPR approach requires one molecule to be immobilized on a sensor chip whereas solution with its binding partner is flowed over the sensor surface. One of the major problems with this technique is, in fact, practically identical to that of bioaffinity chromatography. Upon non-specific, chemical cross-linking to the sensor chip the biological units may lose most of their binding activities and this can significantly affect a signal-to-noise ratio, reproducibility and/or reliability of the SPR results. In the immobilization approach provided herein, a highly specific cross-linking protocol was used. This resulted in nearly 100% of the immobilized Im7 protein molecules retaining full binding activity to the CL7 counterparts. The (CL7/Im7) chromatography approach, thus, may be readily used for construction of reusable (in contrast to disposable chemical chips) Im7-activated SPR biosensors to which various C7-tagged targets can be immobilized under physiological conditions and with high-concentrations.

| SEQUENCES | |
|---|---|
| SEQ ID NO: 1-Wild-type CL7 DNAse domain | |
| KRNKPGKATG KGKPVNNKWL NNAGKDLGSP VPDRIANKLR DKEFKSFDDF RKKFWEEVSK | 60 |
| DPELSKQFSR NNNDRMKVGK APKTRTQDVS GKRTSFELHH EKPISQNGGV YDMDNISVVT | 120 |
| PKRHIDIH | |
| SEQ ID NO: 2-Wild-type CL2 DNAse domain | |
| KRNKPGKATG KGKPVGDKWL DDAGKDSGAP IPDRIADKLR DKEFKNFDDF RKKFWEEVSK | 60 |
| DPDLSKQFKG SNKTNIQKGK APFARKKDQV GGRERFELHH DKPISQDGGV YDMNNIRVTT | 120 |
| PKRHIDIH | |
| SEQ ID NO: 3-Wild-type CL8 DNAse domain | |
| KRNKPGKATG KGKPVGDKWL DDAGKDSGAP IPDRIADKLR DKEFKNFDDF RRKFWEEVSK | 60 |
| DPELSKQFNP GNKKRLSQGL APRARNKDTV GGRRSFELHH DKPISQDGGV YDMDNLRITT | 120 |
| PKRHIDIH | |
| SEQ ID NO: 4-Wild-type CL9 DNAse domain | |
| KRNKPGKATG KGKPVGDKWL DDAGKDSGAP IPDRIADKLR DKEFKSFDDF RKAVWEEVSK | 60 |
| DPELSKNLNP SNKSSVSKGY SPFTPKNQQV GGRKVYELHH DKPISQGGEV YDMDNIRVTT | 120 |
| PKRHIDIH | |
| SEQ ID NO: 5 (72 amino acid linker) | |
| GGTSDGLEVL FQGPNGEDNQ SNHHHHHHHH NQAENSAANN ASDDGNQSSN ESSSDGEQAN | 60 |
| TDSAHEGGTA GS | |

SEQ ID NO: 6 modified CL7 plus linker
GGTSDGLEVLFQGPNGEDNQSNHHHHHHHHNQAENSAANNASDDGNQSSNESSSDGEQANTDSAHEGGTAGSKSNEP
GKATGEGKPVNNKWLNNAGKDLGSPVPDRIANKLRDKEFESFDDFRETFWEEVSKDPELSKQFSRNNNDRMKVGKAP
KTRIQDVSGKRTSFELNHQKPIEQNGGVYDMDNISVVIPKRNIDIEG
SEQ ID NO: 7 modified CL2 plus linker
GGTSDGLEVLFQGPNGEDNQSNHHHHHHHHNQAENSAANNASDDGNQSSNESSSDGEQANTDSAHEGGTAGSKSNEP
GKATGEGKPVGDKWLDDAGKDSGAPIPDRIADKLRDKEFENFDDFRETFWEEVSDPDLSKQFKGSNKTNIQKGKAPF
ARKKDQVGGRERFELNHDKPIEQDGGVYDMNNIRVITPKRNIDIN
SEQ ID NO: 8 modified CL8 plus linker
GGTSDGLEVLFQGPNGEDNQSNHHHHHHHHNQAENSAANNASDDGNQSSNESSSDGEQANTDSAHEGGTAGSKSNEP
GKATGEGKPVGDKWLDDAGKDSGAPIPDRIADKLRDKEFENFDDFRETFWEEVSKDPELSKQFNPGNKKRLSQGLAP
RARNKDTVGGRRSFELNHDKPIEQDGGVYDMDNLRITTPKRNIDIN
SEQ ID NO: 9 modified CL9 plus linker
GGTSDGLEVLFQGPNGEDNQSNHHHHHHHHNQAENSAANNASDDGNQSSNESSSDGEQANTDSAHEGGTAGSEEERE
NNPGKATGEGKPVGDKWLDDAGKDSGAPIPDRIADKLRDKEFESFDDFREAVWEEVSKDPELSKNLNPSNKSSVSKG
YSPFTPKNQQVGGRKVYELNHDQPMWQGGEVYDMDNIRVTTPRRNIEIG
SEQ ID NO: 10 wildtype Im7
MELKNSISDYTEAEFVQLLKEIEKENVAATDDVLDVLLEHFVKITEHPDGIDLIYYPSDN
RDDSPEGIVKEIKEWRAANGKPGFKQG
SEQ ID NO: 11 wildtype Im9
MELKASISDYTEAEFLQLVTTICNADTSSEEELVKLVTHFEEMTEHPSGSDLIYYPKEGD
DDSPSGIVNIVQQWRAANGKSGFKQ
SEQ ID NO: 12 Im7 immobilization unit
MSDKIIHLTDDSFDTDVLKADGAILVDFWAEWCGPCKMIAPILDEIADEYQGKLIVAKLNIDQNPGTAPKYGIRGIP
TLLLFKNGEVAATKVGALSKGQLKEFLDANLAGDTSGTGAEGRRGEEAGKHHHHHHHHGKGAEEGRRGEAGLEVLFQ
GPGQASGTTMGKIEEGKQETWNNGDKGYNGRAEDGGCGGAIEELEEEVRRHQMRLMALQLEEQLMGGCGDVGDMEFR
NSISDYTEEEFVRLLRGIERENVAATDDRLDWMLEHFVEITEHPDGIDLIYYPSDNRDDSPEGIVEEIREWREANGR
PGFKQGGSTDGGDVGEERNRRCEELNEEIEEHRERLRQLEETREECRT
SEQ ID NO: 13: Im9 immobilization unit
MSDKIIHLTDDSFDTDVLKADGAILVDFWAEWCGPCKMIAPILDEIADEYQGKLIVAKLNIDQNPGTAPKYGIRGIP
TLLLFKNGEVAATKVGALSKGQLKEFLDANLAGDTSGTGAEGRRGEEAGKHHHHHHHHGKGAEEGRRGEAGLEVLFQ
GPGQASGTTMGKIEEGKQETWNNGDKGYNGRAEDGGCGGAIEELEEEVRRHQMRLMALQLEEQLMGGCGDVGDMEFR
DSISDYTEEEFLRLVTSICNADTSSEEELVWMVTHFEEITEHPSGSDLIYYPREGD
DDSPSGIVNTVRQWREANGRSGFQQ GSTDGGDVGEERNRRCEELNEEIEEHRERLRQLEETREECRT

1-21. (canceled)

22. A polypeptide comprising a colicin-DNAse domain, wherein the colicin-DNAse domain comprises SEQ ID NO: 1 comprising mutations R2S, K4E, K11E, K45E, K52E, K53T, H99N, S105E, H124N and H128E mutation in SEQ ID NO: 1.

23. The polypeptide of claim 22, wherein the polypeptide further comprises a cleavable polypeptide sequence in operable linkage with the colicin-DNAse domain, wherein the cleavable polypeptide sequence is at least about fifty amino acids in length.

24. The polypeptide of claim 23, wherein the cleavable polypeptide sequence comprises a polypeptide having at least 95 percent identity with SEQ ID NO: 5.

25. The polyptpide of claim 24, wherein the cleavable polypeptide sequence comprises SEQ ID NO: 5

26. The polypeptide of claim 22, wherein the polypeptide comprises SEQ ID NO: 6.

27. The polypeptide of claim 23, further comprising a heterologous protein operably linked to the cleavable polypeptide sequence, wherein the cleavable polypeptide sequence links the heterologous polypeptide with the colicin-DNAse domain.

28. A nucleic acid encoding the polypeptide of claim 22.

29. A vector comprising the nucleic acid of claim 27.

30. A cell comprising the vector of claim 29.

31. A method of purifying a heterologous protein comprising:
a) transfecting in a cell culture medium a cell with a vector, wherein the vector comprises a nucleic acid encoding a first polypeptide under conditions in which the first polypeptide is expressed, wherein the first polypeptide is the polypeptide of claim 25;
b) harvesting the cell culture medium comprising the expressed first polypeptide;
c) lysing the cells to obtain a supernatant comprising the expressed first polypeptide;
d) contacting the supernatant with an affinity matrix comprising a substrate and a second polypeptide, wherein the second polypeptide comprises a colicin immunity protein with one or more mutations,
e) washing the matrix to remove biological molecules non-specifically bound to the first expressed polypeptide and the matrix;
f)) eluting the heterologous protein from the matrix, comprising enzymatically cleaving the heterologous protein from the first polypeptide.

32. The method of claim 31, wherein the colicin immunity protein comprises SEQ ID NO: 10 comprising mutations L3F, K4R, A13E, Q17R, K20R, E21G, K24R, V33R, V36W, L37M, K43E, K70E, K73R, A77E and K81R.

33. A method of purifying a heterologous protein comprising:
a) transfecting in cell culture medium a cell with a vector comprising a nucleic acid encoding a first polypeptide, wherein the first polypeptide comprises the polypeptide of claim 25 under conditions in which the first polypeptide comprising the heterologous protein is expressed;
b) harvesting the cell culture medium comprising the expressed first polypeptide comprising a heterologous protein;
c) contacting the harvested cell culture medium with an affinity matrix, wherein the affinity matrix comprises a substrate and a second polypeptide comprising a colicin immunity protein with one or more mutations;
d) washing the matrix to remove biological molecules non-specifically bound to the expressed first polypeptide comprising a heterologous protein and the matrix;
e) eluting the heterologous protein from the matrix, comprising enzymatically cleaving the heterologous protein from the first polypeptide.

34. The method of claim 33, wherein the colicin immunity protein comprises SEQ ID NO: 10 comprising mutations L3F, K4R, A13E, Q17R, K20R, E21G, K24R, V33R, V36W, L37M, K43E, K70E, K73R, A77E and K81R.
